(19) United States
(12) Patent Application Publication
(10) Pub. No.: US 2025/0255976 A1
(43) Pub. Date: Aug. 14, 2025

(54) CATIONIC BIOMATERIALS AMELIORATE OBESITY-ASSOCIATED CHRONIC INFLAMMATION AND FOCAL ADIPOSITY

(71) Applicant: THE TRUSTEES OF COLUMBIA UNIVERSITY IN THE CITY OF NEW YORK, New York, NY (US)

(72) Inventors: Kam W. LEONG, New York, NY (US); Li QIANG, New York, NY (US); Tianyu LI, New York, NY (US); Baoding HUANG, New York, NY (US); Qianfen WAN, New York, NY (US)

(73) Assignee: THE TRUSTEES OF COLUMBIA UNIVERSITY IN THE CITY OF NEW YORK, New York, NY (US)

(21) Appl. No.: 19/171,066

(22) Filed: Apr. 4, 2025

Related U.S. Application Data
(63) Continuation of application No. PCT/US23/75966, filed on Oct. 4, 2023.
(60) Provisional application No. 63/413,141, filed on Oct. 4, 2022.
(60) Provisional application No. 63/383,007, filed on Nov. 9, 2022.

(51) Int. Cl. A61K 47/64; A61K 9/00; A61K 31/785; A61K 47/69; A61P 3/08; A61P 29/00

(52) U.S. Cl. CPC A61K 47/643; A61K 9/0019; A61K 31/785; A61K 47/6921; A61P 3/08; A61P 29/00

(57) ABSTRACT

Compositions, and methods of treating a subject using the compositions, for treating obesity, reducing body weight, treating chronic inflammation associated with obesity, reducing focal adiposity, and improving metabolism. The compositions include a complex comprising cationic PAMAM generation 3 (P-G3) and human serum albumin (HSA). The complex may be a cfRNA or cfDNA scavenger.

### RELATED APPLICATIONS

This application is a continuation of International Application No. PCT/US2023/75966, filed Oct. 4, 2023 to Leong et al., titled CATATONIC BIOMATERIALS AMELIORATE OBESITY-ASSOCIATED CHRONIC INFLAMMATION AND FOCAL ADIPOSITY, which claims the benefit of U.S. provisional patent application 63/413,141, filed Oct. 4, 2022 to Leong, et al., titled CATIONIC BIOMATERIALS AMELIORATE OBESITY-ASSOCIATED CHRONIC INFLAMMATION AND FOCAL ADIPOSITY, and the benefit of U.S. provisional patent application 63/383,007, filed Nov. 9, 2022 to Leong, et al., titled CATIONIC BIOMATERIALS AMELIORATE OBESITY-ASSOCIATED CHRONIC INFLAMMATION AND FOCAL ADIPOSITY, the entirety of the disclosures of which are hereby incorporated by this reference.

### STATEMENT REGARDING FEDERALLY SPONSORED RESEARCH

This invention was made with government support under Grant No. AR073935 awarded by the National Institutes of Health. The government has certain rights in the invention.

### TECHNICAL FIELD

The present subject matter relates to compositions, and methods for using the compositions, for treating obesity, reducing body weight, and improving metabolic functions in a subject. In particular, the compositions include a complex comprising cationic PAMAM generation 3 (P-G3) and human serum albumin (HSA).

### BACKGROUND

As a surging public health crisis, obesity and overweight create heavy burdens for the health care systems worldwide. Over the past half century, their rates have tripled and will threaten over 2.5 billion adults by 2025 [1, 2]. The high prevalence is particularly alarming given that obesity and overweight predispose affected individuals to a number of serious comorbidities contributed by the accompanying chronic inflammation, such as type 2 diabetes mellitus (T2DM), cardiovascular diseases, osteoarthritis, and various cancers, all of which are major contributors to morbidity and mortality in modern society. However, few options exist for tackling chronic inflammation in obesity or inhibiting depot-specific adiposity.

A major mechanism underpinning the link between obesity and associated comorbidities is sterile, low-grade inflammation, namely, chronic inflammation, which occurs in multiple tissues, especially white adipose tissue (WAT). Obesity induces a series of pathological events in WAT that trigger chronic inflammation, including macrophage infiltration [3, 4] and subsequent proinflammatory cytokine and chemokine secretion [5, 6], hypoxia due to decreased vascular density [7-9], cell death [10], mitochondrial dysfunction [11], endoplasmic reticulum (ER) stress [12-15], etc. The inflammatory state further impairs the metabolic functions of WAT in glucose uptake and lipid storage. As a consequence, excess lipids are deposited into ectopic tissues, such as the liver and muscle, and cause lipotoxicity and worsen insulin resistance, further exacerbating inflammation in WAT in a feed-forward manner. In this regard, obesity and its comorbidities are appreciated as chronic inflammatory diseases. Therefore, improving inflammatory profiles would likely decrease the risk of obesity-associated metabolic disorders [16]. However, despite the tremendous success of numerous anti-inflammatory drugs, the treatment of chronic inflammation in obesity has thus far been lacking [17-22].

Various cationic biomaterials have produced a therapeutic outcome in treating acute inflammation in diverse animal disease models, including acute liver failure [23], trauma [24, 25], lupus [26], rheumatoid arthritis [27, 28], spinal cord injury [29], inflammatory bowel disease [30], and bacterial sepsis [31-33]. However, they have never been tested in metabolic diseases despite this promising anti-inflammatory effect. Polyamidoamine (PAMAM) is the most well-characterized class of dendrimer, possessing a symmetric and highly branched molecular structure. Other than its conventional use as a carrier or for gene delivery, cationic PAMAM can also neutralize negatively charged pathogens [34]. An important class of these pathogens is cell-free nucleic acids (cfNAs), which are released by dead and dying cells but can also be contributed by the gut microbiome, viruses, or even food. These cfNAs are well-established ligands for Toll-like receptors (TLRs), a major class of regulators mediating the innate immune response.

### SUMMARY

Polycationic polyamidoamine (PAMAM) treatment can improve both aspects of obesity—chronic inflammation and local adiposity. Because the plasma cell-free RNA (cfRNA) level is elevated in obese subjects, the cationic PAMAM generation 3 (P-G3) scavenger may be applied to treat diet-induced obese (DIO) mice—specifically, accumulated fat in the subject. Administration of P-G3, including without limitation through intraperitoneal delivery, may alleviate the chronic inflammation in DIO mice and reduce body weight, resulting in improved metabolic functions. To further enhance the applicability of P-G3, we complexed the P-G3 with human serum albumin (HSA) to attain a sustained release, which showed consistent benefits in treating DIO mice. Local injection of HSA-PG3 into subcutaneous fat completely restricted the distribution of the complex within the targeted depot and reduced focal adiposity. Our study illuminates a promising cationic strategy to ameliorate chronic inflammation in obesity and target local adiposity.

In one aspect, provided herein is a complex for treating accumulated fat in a subject, comprising human serum albumin (HSA) and cationic polyamidoamine generation 3 (P-G3) in complex with the HSA. The complex may be formed in microspheres and may be configured for treating obesity, reducing body weight, and/or improving metabolic functions in the subject. The microspheres may have a diameter between 0.5 micrometers and 500 micrometers. The complex may have an HSA:P-G3 mass ratio of at least 5:1. In some embodiments, the complex has an HSA:P-G3 mass ratio of about 10:1. The microspheres attain controlled release of the P-G3 into the subject.

In one aspect, provided herein is a method of treating accumulated fat in a subject, comprising administering to the subject a therapeutically effective amount of (a) the complex described above or (b) a pharmaceutically acceptable salt of P-G3 and at least one pharmaceutically acceptable excipient. The administration may comprise enteral administration of the complex to the subject. The administration may comprise parenteral administration of the complex to the subject. The administration may comprise local injection into subcutaneous fat of the subject. The administration may comprise a plurality of injections. The method may treat or reduce focal adiposity in the subject. The method may ameliorate chronic inflammation associated with obesity in the subject. The method may result in metabolic improvements in the subject. The complex may scavenge cfDNA. The complex may scavenge cfRNA.

In one aspect, provided herein is a method of treating accumulated fat in a human subject in need thereof, comprising administering into the accumulated fat a therapeutically effective amount of P-G3 and/or HSA-PG3 or a pharmaceutically acceptable salt thereof and at least one pharmaceutically acceptable excipient, wherein the accumulated fat results from or causes one or more of excess axillary fat, lateral periaxillary fat, pre axillary fat, post axillary fat, anterior periaxillary fat, posterior periaxillary fat, fat on the upper back, bra fat, back of arm fat, fat on the anterolateral flank, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including, but not limited to periumbilical fat, fat above periumbilical area, fat below periumbilical area, or any combination of two or more thereof), excess fat on the buttocks, mons pubis fat, excess fat around the ankles, fat on the upper back of the thigh, excess fat on the foot, pseudogynocomastia fat, lipoma, lipodystrophy (such as Dunning-type lipodystrophy), lipomatosis such as familial multiple lipomatosis, post-liposuction fat deposits, and obstructive sleep apnea.

In another aspect, provided herein is a method of treating accumulated fat in a human subject in need thereof, comprising administering into the accumulated fat a therapeutically effective amount of P-G3 and/or HSA-PG3 or a pharmaceutically acceptable salt thereof and at least one pharmaceutically acceptable excipient, wherein the accumulated fat results from or causes one or more of bra fat, back of arm fat, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including periumbilical fat), excess fat on the buttocks, mons pubis fat, excess fat around the ankles, lipoma, lipodystrophy (such as Dunning-type lipodystrophy), lipomatosis such as familial multiple lipomatosis, post-liposuction fat deposits, and obstructive sleep apnea.

In another aspect, provided herein is a method of reducing accumulated fat in a human subject in need thereof, comprising administering into the accumulated fat a therapeutically effective amount of P-G3 and/or HSA-PG3 or a pharmaceutically acceptable salt thereof and at least one pharmaceutically acceptable excipient, wherein the accumulated fat results from or causes one or more of excess axillary fat, lateral periaxillary fat, pre axillary fat, post axillary fat, anterior periaxillary fat, posterior periaxillary fat, fat on the upper back, bra fat, back of arm fat, fat on the anterolateral flank, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including, but not limited to periumbilical fat, fat above periumbilical area, fat below periumbilical area, or any combination of two or more thereof), excess fat on the back or buttocks, mons pubis fat, excess fat around the ankles, pseudogynocomastia fat, lipoma, lipodystrophy (such as Dunning-type lipodystrophy), lipomatosis such as familial multiple lipomatosis, post-liposuction fat deposits, and obstructive sleep apnea.

In another aspect, provided herein is a method of reducing accumulated fat in a human subject in need thereof, comprising administering into the accumulated fat a therapeutically effective amount of P-G3 and/or HSA-PG3 or a pharmaceutically acceptable salt thereof and at least one pharmaceutically acceptable excipient, wherein the accumulated fat results from or causes one or more of bra fat, back of arm fat, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including periumbilical fat), excess fat on the back or buttocks, mons pubis fat, excess fat around the ankles, lipoma, lipodystrophy (such as Dunning-type lipodystrophy), lipomatosis such as familial multiple lipomatosis, post-liposuction fat deposits, and obstructive sleep apnea.

In some embodiments, the P-G3 and/or HSA-PG3 is administered by injection. In some embodiments, the P-G3 and/or HSA-PG3 is administered by subcutaneous injection. In some embodiments, the P-G3 and/or HSA-PG3 is administered by a plurality of injections. In some embodiments, the P-G3 and/or HSA-PG3 is administered by a plurality of subcutaneous injections.

The polycationic material PAMAM generation 3 (P-G3) discussed herein displays an anti-obesity effect and alleviates chronic inflammation in obesity. Additionally, sustained release of the P-G3 into the subject may be attained by complexing the P-G3 with human serum albumin (HSA). HSA-PG3 complex may be restricted to the targeted fat depot via local delivery and may reduce focal adiposity.

### BRIEF DESCRIPTION OF THE DRAWINGS

Implementations will hereinafter be described in conjunction with the appended and/or included DRAWINGS, where like designations denote like elements.

FIG. 1 is a graphic representation of administration of P-G3 to a subject. Systemic intraperitoneal administration of P-G3 reduces adiposity, alleviates inflammation, and improves metabolic functions in diet-induced obese mice. When complexed with HSA, the resultant microspheres attain controlled release of P-G3 and inhibit focal adiposity in subcutaneous iWAT fat depot via local injection.

FIG. 2A is a schematic diagram of the experimental design. Plasma cell-free RNA is increased in obese mice and activates TLR3. For FIGS. 2A-2P, * p<0.05, ** p<0.01, *** p<0.001, **** p<0.0001. Data are presented as the mean±SEM.

FIG. 2B illustrates the activity of TLR3 when TLR-HEK293T reporter cells were treated with the plasma collected from lean or obese mice.

FIG. 2C illustrates the activity of TLR4 when TLR-HEK293T reporter cells were treated with the plasma collected from lean or obese mice.

FIG. 2D illustrates the activity of TLR8 when TLR-HEK293T reporter cells were treated with the plasma collected from lean or obese mice.

FIG. 2E illustrates the activity of TLR9 when TLR-HEK293T reporter cells were treated with the plasma collected from lean or obese mice.

FIG. 2F illustrates the activity of TLR3-HEK293T reporter cells after incubation with obese plasma with or without TLR3 inhibitor or pretreated with RNase I.

FIG. 2G illustrates the activity of TLR8-HEK293T reporter cells after incubation with obese plasma with or without TLR8 inhibitor.

FIG. 2H illustrates cfRNA level in the plasma from lean and DIO C57BL/6 mice.

FIG. 2I illustrates cfRNA levels in the plasma from obesity-resistant 129/Sv mice that received a chow diet or HFD feeding.

FIG. 2J illustrates the activity of TLR3-HEK293T reporter cells after incubation with the plasma from 129/Sv mice fed a chow diet or HFD.

FIG. 2K illustrates the activity of TLR3 after treatment with plasma from wild-type (WT) or PPARα KO mice on HFD feeding.

FIG. 2L illustrates the activity of TLR3-HEK293T reporter cells after treatment with liver RNA (RNA-L) from obese or lean mice.

FIG. 2M illustrates the activity of TLR3-HEK293T reporter cells after treatment with obese liver RNA in the presence of TLR3 inhibitor, RNase I, or P-G3.

FIG. 2N illustrates the activity of TLR3 KO HEK-Dual Null reporter cells with or without the addition of obese liver RNA.

FIG. 2O illustrates the ELISA determination of TNF-α levels in the RAW264.7 cells macrophage culture medium at the indicated treatments.

FIG. 2P illustrates the activity of TLR3-HEK293T reporter cells after incubation with the plasma from P-G3-treated obese mice.

FIG. 3A is a schematic diagram of the experimental design. Male C57BL/6 mice were fed HFD for 20 weeks to induce obesity. P-G3 displays anti-obesity effect in DIO mouse model. For FIGS. 3A-3L, * p<0.05, ** p<0.01, *** p<0.001 for vehicle control (n=7) vs. the P-G3 (n=7) group by 2-tailed Student's t-test. Data are presented as the mean SEM.

FIG. 3B illustrates body weight before and after P-G3 treatment.

FIG. 3C illustrates tissue weights at sacrifice.

FIG. 3D illustrates a histological analysis (H & E staining) of the eWAT.

FIG. 3E illustrates eWAT gene expression.

FIGS. 3F-3I illustrate calorimetric analyses of mice with or without P-G3 treatment at ambient temperature, including food intake (3F), activity (3G), oxygen consumption (3H), and respiratory exchange ratio within 1 dark/light cycle (3I).

FIG. 3J illustrates a glucose tolerance test after 8 weeks of P-G3 treatment.

FIG. 3K illustrates liver gene expression.

FIG. 3L illustrates a histological analysis (H & E staining) of the liver.

FIGS. 4A-4D illustrate the anti-inflammatory function of P-G3 in DIO mice. * p<0.05, **p<0.01 for the P-G3 treatment groups vs. the vehicle control group. by 2-tailed Student's t test. Data are presented as the mean±SEM.

FIG. 4A illustrates cfRNA levels in the plasma of male DIO mice after 8-wk P-G3 treatment (n=7, 7).

FIG. 4B illustrates the activity of TLR3-HEK293T reporter cells after exposure to the plasma from mice with or without 8-wk P-G3 treatment (n=7, 7).

FIG. 4C illustrates inflammatory gene expression in eWAT from mice in FIG. 2A (n=6, 6).

FIG. 4D illustrates gene expression of inflammatory markers in eWAT (n=5, 3, 5). After 4 weeks of HFD feeding, mice received vehicle or a single dose of P-G3 (10 μg/g BW, IP), and were collected tissues at Day 3 or Day 7 post-injection.

FIGS. 5A-5N illustrate complexing P-G3 with HSA and characterizations. ** p<0.01, *** p<0.001 for treatment group vs vehicle control group by 2-tailed Student's t-test. Data were represented as mean±SEM.

FIGS. 5A-5D illustrate optimization of HSA-PG3 complexes with different HSA:P-G3 mass ratios based on hydrodynamic diameter (5A), count rate (5B), polydispersity index (PDI) (5C), and zeta potential (n=3) (5D).

FIG. 5E illustrates size distribution histograms of the optimized HSA-PG3 complex with an HSA:P-G3 mass ratio of 10:1.

FIG. 5F illustrates a TEM picture showing the morphology of the optimized HSA-PG3 complex. Scale bar, 2 μm.

FIGS. 5G and 5H illustrate stability of HSA-PG3 complex in H₂O and cell culture media (RPMI with 5% FBS) based on relative size change (5G) and relative zeta potential change (n=3) (5H).

FIG. 5I illustrates a release profile of HSA-PG3 in PBS (n=3).

FIG. 5J illustrates a comparison of the DNA binding efficiency of P-G3 and HSA-PG3 at different polymer:DNA ratios (n=3, 3).

FIG. 5K illustrates the RNA binding efficiency of P-G3 or HSA-PG3 (n=3, 3).

FIG. 5L illustrates the activity of TLR3-HEK293T reporter cells after incubation with 2 μg/mL Poly(I:C) with or without P-G3 or HSA-PG3 (1 μg/mL) pretreatment (n=3, 3).

FIG. 5M illustrates 3T3-L1 cell viability upon exposure to different concentrations of P-G3 or HSA-PG3 (n=6, 6).

FIG. 5N illustrates comparable effects of HSA-PG3 and P-G3 on the inhibition of lipogenesis in 3T3-L1 adipocytes on Day 6 of differentiation (n=3, 3).

FIGS. 6A-6J illustrate treatment of diet-induced obese mice by HSA-PG3. * p<0.05, ** p<0.01, and *** p<0.001 for vehicle control (n=7) vs. HSA-PG3 group (n=7 for metabolic characterizations, n=6 for qPCR because of the loss of one sample in processing) by 2-tailed Student's t test. Data are presented as the mean±SEM.

FIG. 6A illustrates a comparison of the biodistribution of Cy5-labeled HSA-PG3 or P-G3 in DIO mice via the IP delivery. Tissue fluorescent signals were determined by using an IVIS Optical Imager at Day 1, Day 8, and Day 15 post-injection of 200 μg polymers. iWAT: inguinal WAT; eWAT: epididymal WAT; rWAT: retroperitoneal WAT; BAT: brown adipose tissue.

FIG. 6B illustrates a schematic diagram of the experimental design. Male C57BL/6 mice were treated with HSA-PG3 or PBS vehicle control at the start of HFD feeding.

FIG. 6C illustrates the body weight curve during treatment.

FIG. 6D illustrates the body weight change during treatment.

FIG. 6E illustrates tissue weights at sacrifice.

FIG. 6F illustrates a histological analysis (H & E staining) in the eWAT (n=7, 6).

FIGS. 6G and 6H illustrate eWAT gene expression.

FIGS. 6I and 6J illustrate a glucose tolerance test and an insulin tolerance test, respectively.

FIGS. 7A-7H illustrate focal subcutaneous adiposity reduction by HSA-PG3. * p<0.05, ** p<0.01, *** p<0.001 for vehicle control (n=6) vs. the HSA-PG3 group (n=6) by paired Student's t test. Data are presented as the mean±SEM.

FIG. 7A illustrates the fluorescent signal in tissues imaged using an IVIS system at Day 3 after 50 μg of Cy5-labeled HSA-PG3 injection on one inguinal side. iWAT: inguinal WAT; eWAT: epididymal WAT; rWAT: retroperitoneal WAT; BAT: brown adipose tissue, tAT: thigh adipose tissue (adjacent to iWAT). Non-treated (NT, n=1), HSA-PG3 group (n=3).

FIG. 7B illustrates a schematic diagram of the experimental design. DIO male C57BL/6 mice were injected 50 μg HSA-PG3 and PBS on each side iWAT weekly.

FIG. 7C illustrates photos of the mice and iWAT after 5 weeks of HSA-PG3 injection on one inguinal side and PBS on the other side.

FIG. 7D illustrates iWAT weights of the HSA-PG3-treated or control side at sacrifice.

FIG. 7E illustrates a histological analysis (H & E staining) in iWAT with or without HSA-PG3 treatment.

FIGS. 7F-7H illustrate qPCR expression of lipogenic (7F), adipogenic (7G), and inflammatory genes (7H).

FIGS. 8A-8D illustrate characterizations of P-G3.

FIG. 8A illustrates colocalization of Cy5-labeled P-G3 (red color) with DAPI (staining for nuclei, blue color) and Caveolin-1 (staining for adipocyte cell membrane, green color) in eWAT from mice at 1-day post-injection.

FIG. 8B illustrates western blotting of obese eWAT proteins after 8 weeks of P-G3 treatment. * p<0.05, ** p<0.01, *** p<0.001 for vehicle control vs. P-G3 group by 2-tailed Student's t-test (n=7, 7).

FIG. 8C illustrates C3H10T1/2 cell viability upon exposure to different concentrations of P-G3 (n=3).

FIG. 8D illustrates the blunted effect of HSA (100 μg/mL) on 3T3-L1 adipocytes' gene expression on Day 6 of differentiation using PBS as the vehicle control (n=3). Data were represented as mean±SEM.

FIGS. 9A-9C illustrate the biodistribution of HSA-PG3.

FIG. 9A illustrates the quantification of P-G3 or HSA-PG3 tissue distribution overtime in FIG. 6A. Vehicle (n=1), P-G3 or HSA-PG3 group (n=2).

FIGS. 9B and 9C illustrate that the biodistribution of HSA-PG3 is delivery route-dependent. 200 g Cy5-labeled HSA-PG3 was IP or IV injected into mice. At Day 3 post-injection, fluorescent signals in tissues were imaged (9B) and quantified (9C). PBS group (n=1), HSA-PG3 treatment groups (n=1).

FIGS. 10A-10F illustrate the restriction of HSA-PG3 to iWAT by local injection.

FIGS. 10A-10C illustrate results when 50 g Cy5-labeled HSA-PG3 was injected into one side of inguinal WAT depot. FIG. 10A shows the imaging of tissue fluorescent signal in mice sacrificed at 7-day post-injection. FIG. 10B illustrates the quantification of fluorescent signals at 3-day post-injection. FIG. 10C illustrates the quantification of fluorescent signals at 7-day post-injection.

FIGS. 10D-10F illustrate local iWAT injection of 500 g has into male DIO mice.

FIG. 10D illustrates no effect on iWAT mass (n=7, 7). FIGS. 10E-10F illustrate Q-PCR determination of the expression of key lipogenic genes (10E) and adipogenic markers (1° F.) (n=6, 6). * p<0.05 for vehicle control side vs. the HSA side by paired Student's t-test. Data are presented as the mean±SEM.

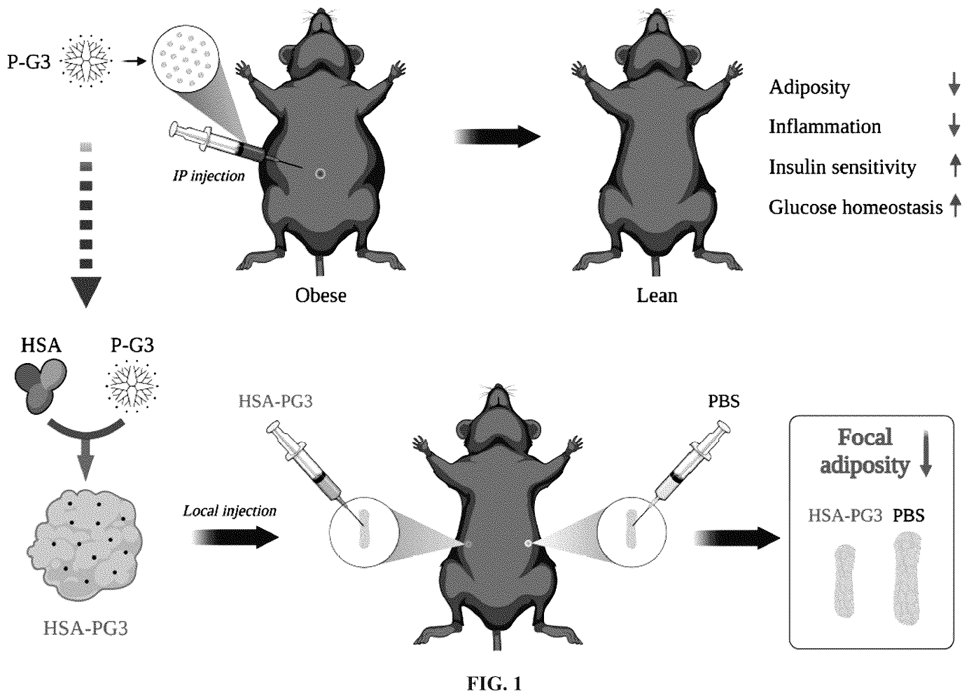

### DETAILED DESCRIPTION

It is noted that, as used in this specification and the appended claims, the singular forms “a”, “an”, and “the” include plural references unless the context clearly dictates otherwise.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

The present disclosure is related to a complex for treating accumulated fat in a subject that comprises cationic polyamidoamine generation 3 (P-G3). The complex is configured for treating obesity, reducing body weight, and/or improving metabolic functions in the subject. In some embodiments, the P-G3 is complexed with human serum albumin (HSA). In some embodiments, the complex is formed in microspheres. These microspheres may attain controlled release of the P-G3 into the subject.

The present disclosure is also related to a method of treating accumulated fat in the subject. This method comprises administering a therapeutically effective amount of the complex to the subject, or comprises administering a pharmaceutically acceptable salt of P-G3 and at least one pharmaceutically acceptable excipient to the subject. This method is explained in more detail below.

The cationic scavenging complexes of the present invention, or pharmaceutically acceptable salts thereof, can be administered to the patient via any systemic or local route such that effective levels are achieved in, for example, the bloodstream or in the target tissue. The optimum dosing regimen will depend, for example, on the cationic complex, the patient, and the effect sought. Typically, the cationic complex will be administered enterally or parenterally, including without limitation orally, sublingually, transdermally, topically or cutaneously, subcutaneously (SC), intravenously (IV), intramuscularly (IM), or intraperitoneally (IP). The cationic complex may also be administered, for example, directly to a target tissue site, for example, directly to subcutaneous fat in the subject.

The cationic scavenging complexes of the present invention, or pharmaceutically acceptable salts thereof, can be formulated with one or more carriers, diluents, excipients, additional treating agents, or other appropriate component to yield a suitable pharmaceutical composition. The precise nature of the compositions of the invention will depend, at least in part, on the nature of the desired treatment and the route of administration. Optimum dosing regimens can be readily established by one skilled in the art and can vary with, for example, the cationic complex, the patient, and the particular effect sought, including whether the desired effect is general or targeted to a particular area of the patient.

The ratio of primary components in the complex, such as the HSA:PG3 ratio, can vary significantly while maintaining efficacy in treating focal adiposity and chronic inflammation associated with obesity. For example, one embodiment involves preparation and use of a complex with an HSA:P-G3 ratio of 10:1. Other successful embodiments include ratios of, for example, about 3:1 to about 30:1 HSA:P-G3. One preferred embodiment is a ratio of about 5:1 to about 20:1 HSA:P-G3. Preferred ratios are greater than 1:1, and less than 40:1, HSA:P-G3. Accordingly, the ratio may be great than 1:1, or about 2:1; 3:1; 4:1; 5:1; 6:1; 7:1; 8:1; 9:1; 10:1; 11:1; 12:1; 13:1; 14:1; 15:1; 16:1; 17:1; 18:1; 19:1; 20:1; 21:1; 22:1; 23:1; 24:1; 25:1; 26:1; 27:1; 28:1; 29:1; 30:1; 31:1; 32:1; 33:1; 34:1; 35:1; 36:1; 37:1; 38:1; 39:1; or less than 40:1 HSA:P-G3.

It will be appreciated that the treatment methods of the present invention are useful in the fields of both human medicine and veterinary medicine. Thus, the patient (subject) to be treated can be a mammal, preferably a human. For veterinary purposes the subject can be, for example, a farm animal such as a cow, pig, horse, goat, or sheep, or a companion animal such as a dog or cat.

In the studies leading to the present disclosure, strong activation of Toll-like receptors 3 and 8 (TLR3 and 8) by the plasma from diet-induced obese (DIO) mice was detected, which was attributed to increased cell-free RNA (cfRNA). Because cationic PAMAM generation 3 (P-G3) is able to efficiently scavenge cfRNA, the potential of this compound to mitigate chronic inflammation was investigated, ultimately achieving metabolic improvements in obesity. P-G3 displayed a potent anti-obesity effect, accompanied by improved metabolic status. Finally, we complexed P-G3 with human serum albumin (HSA) to improve the safety profile via a sustained release. Beyond the milder anti-obesity effect of HSA-PG3 by systemic delivery, local delivery of HSA-PG3 to the subcutaneous fat depot restricted the distribution within this depot and specifically reduced the focal adiposity. Therefore, these findings open up new avenues to ameliorate chronic inflammation and local adiposity via polycationic materials.

Harnessing chronic inflammation and focal fat reduction are two prominent challenges in obesity treatment. In this study, polycationic material is applied to alleviate chronic inflammation in obesity, resulting in metabolic improvements, including obesity inhibition. Furthermore, by uncovering the highly restricted local fat distribution of P-G3 in complexes with HSA, we trailblaze a new cationic strategy for treating focal adiposity. These findings signify novel functions and applications of cationic biomaterials in tackling metabolic diseases.

Numerous mechanisms underlie chronic inflammation in obesity and the detrimental development of obesity comorbidities, such as immune cell infiltration and activation, inflammatory cytokine production, lipid toxicity, and microbiome-derived endotoxin. In understanding the triggers of chronic inflammation, the activation of TLRs, the pivotal gatekeepers of the innate immune response, by the plasma of obese mice were screened. Positive responses by TLR3 and TLR8 were found, both of which are receptors of dsRNA and ssRNA, respectively. Although most RNA in mammalian cells is single-stranded, RNA is known to form intra-strand or inter-strand double-stranded structures [36], which may serve as endogenous ligands of TLR3 [43]. The lack of response by TLR4 and TLR9 reporter cells could be explained by the different assay systems (source of stimulants, model cells, sensitivity, etc.) rather than excluding their ligands from the stimulant pool. Nevertheless, the data collected from TLR3 overexpression and KO cells, TLR3/8 inhibitors, and RNA-specific nucleases collectively demonstrate that cfRNA is a pathogen in obesity to intensify inflammation via TLR3/8 signaling and could be used as a surrogate marker of chronic inflammation in obesity. The sources are likely to be apoptotic cells or damaged cells in various tissues from the stress of weight gain, such as the liver, given that total RNA isolated from the obese liver is more potent in inducing TLR3. Moreover, since the minimum length of extracted RNA using the kit was over 200 nt, the possible effect of small RNAs was filtered out. Furthermore, considering cfDNA-activated macrophages through TLR9 are involved in adipose tissue inflammation and insulin resistance [44], P-G3 could unanimously scavenge both cfRNA and cfDNA to alleviate inflammation in obesity.

Numerous mechanisms underlie chronic inflammation in obesity and the detrimental development of obesity comorbidities, such as immune cell infiltration and activation, inflammatory cytokine production, lipid toxicity, and microbiome-derived endotoxin. In understanding the triggers of chronic inflammation, we screened the activation of TLRs, the pivotal gatekeepers of the innate immune response, by the plasma of obese mice and found positive responses by TLR3 and TLR8, both of which are receptors of dsRNA and ssRNA, respectively. Although most RNA in mammalian cells is single-stranded, RNA is known to form intra-strand or inter-strand double-stranded structures [36], which may serve as endogenous ligands of TLR3 [43]. The lack of response by TLR4 and TLR9 reporter cells could be explained by the different assay systems (source of stimulants, model cells, sensitivity, etc.) rather than excluding their ligands from the stimulant pool. Nevertheless, the data collected from TLR3 overexpression and KO cells, TLR3/8 inhibitors, and RNA-specific nucleases collectively demonstrate that cfRNA is a pathogen in obesity to intensify inflammation via TLR3/8 signaling and could be used as a surrogate marker of chronic inflammation in obesity. The sources are likely to be apoptotic cells or damaged cells in various tissues from the stress of weight gain, such as the liver, given that total RNA isolated from the obese liver is more potent in inducing TLR3. Moreover, since the minimum length of extracted RNA using our kit was over 200 nt, the possible effect of small RNAs was filtered out. Furthermore, considering cfDNA-activated macrophages through TLR9 are involved in adipose tissue inflammation and insulin resistance [44], P-G3 could unanimously scavenge both cfRNA and cfDNA to alleviate inflammation in obesity.

The scavenging effect of P-G3 on cfRNA mainly depends on its cationic surface that interacts with negatively charged molecules. This cationic nature of PAMAM function is linked to the main concerns regarding its toxicity, which is believed to arise from the binding to and destabilizing of anionic cell membranes, eventually leading to cell lysis [45]. Cytotoxicity largely depends on the concentration, charge density, and structure and varies across models [46]. The half maximal inhibitory concentration (IC50) of P-G3 to induce significant mortality in zebrafish embryos is 2 mg/mL [47], whereas the same polycation shows an IC50 of over 10 mg/mL in L929 mouse fibroblasts [48]. Higher generations of PAMAM have better efficiency in gene or drug delivery but also higher cytotoxicity. Hence, a balance between its efficacy and cytotoxicity should be carefully evaluated when applied to obesity treatment. In the present study, the chosen dose of 10 μg/mL P-G3 did not lead to viability change in the 3T3-L1 and C3H10T1/2 cell lines, nor did it cause any noticeable toxicity in vivo, such as changes in food intake and feeding behavior, locomotion activity, and lean body mass. Therefore, the anti-obesity effect of P-G3 is likely not due to the complications or consequences of toxicity. Furthermore, neither the enriched distribution of P-G3 in eWAT nor the local injection of HSA-PG3 into iWAT caused inflammatory macrophage infiltration. In contrast, P-G3 stimulated an anti-inflammatory response to alleviate chronic inflammation in the obese adipose tissue microenvironment.

Nonetheless, there are safety concerns with using polycationic PAMAM in treating obesity. The pharmacokinetic and pharmacodynamic properties and efficacy can be improved by modifying the exterior of PAMAM with various chemicals or biomolecules or further fabricating them into nanoparticles [49, 50]. For example, we have previously shown that cationic nanoparticles can be more efficiently distributed into inflamed tissues to more effectively block the TLR9 pathway [31]. Here, we introduced a new manipulation of PAMAM by forming microspheres in complex with HSA. Albumin is produced by the liver and is highly present in the serum and is recognized as an excellent drug delivery system due to advantages such as high drug loading capacity, nontoxicity, and low immunogenicity. It can be easily prepared into well-defined sizes from nanoparticles to microspheres for different application contexts. Albumin microspheres ranging from several micrometers to several hundred micrometers hold benefits such as the controlled release of cargo, specific targeting of a desired tissue or organ, protection of the payload from degradation or clearance, and increased biocompatibility [51-53]. In our study, HSA was employed as the carrier of P-G3 to form microspheres with a diameter of approximately 1 μm. This modification improved the adipose tissue distribution and retention of P-G3, showing a potent and highly specific effect on reducing focal adiposity. Future engineering of polycationic materials with albumin may further improve the potency and reduce the administration frequency in treating adiposity.

The preferential enrichment of systemic IP delivery into visceral fat depots, together with the specific distribution of local injection into focal fat depots, is an intriguing characteristic of polycationic PAMAM. In a parallel study, we demonstrate that the cationic charge is required for interacting with the highly negatively charged extracellular matrix of adipose tissue, accounting for the adipose-specific distribution. The cationic charge-dependent distribution to adipose tissue can be leveraged to address the challenges of obesity treatment. PAMAM has been extensively studied as delivery vehicles, and the globular interior of high-generation PAMAM is suitable for loading bioactive compounds [54]. Using P-G3 to deliver fat-manipulating reagents and gene therapies into a targeted location may achieve the additive benefit of inhibiting adiposity. Further, the strong suppression of the lipogenesis program by P-G3 may facilitate the discovery of novel mechanisms to manipulate adipocytes. In summary, the present study documents the potential of polycationic materials in treating metabolic diseases, with the dual benefits of alleviating chronic inflammation and inhibiting adiposity, particularly in a depot-specific manner.

This disclosure is based on a study that involved particular methods implemented to ascertain the usefulness of P-G3 in treating accumulated fat in a subject. The following paragraphs explain these methods.

### 3. Materials and Methods

### 3.1. Detection of Plasma RNA Content

The RNA concentration was detected with a Quant-iT RNA assay kit (Q33140, Thermo Fisher Scientific) following the manufacturer's instructions. Murine plasma was harvested from anticoagulated blood by centrifugation at 6000 rpm for 6 min. RNA detection working solution was freshly prepared by diluting RNA reagent in RNA buffer (volume ratio 1:200). 10 μL of E. coli rRNA standards or 2 μL of murine plasma sample was added to separate wells in a 96-well plate (3915, Corning Costar) loaded with 200 μL of working solution and mixed well. The fluorescence was measured using a microplate reader at excitation/emission 620/670 nm to determine the RNA concentration according to the standard curve.

### 3.2. Cell Culture

Stable TLR3-, 4-, 8-, and 9-overexpressing HEK-Blue and TLR3 knockout (KO) HEK-Dual Null cells (Invivogen) were propagated in high-glucose Dulbecco's modified Eagle's medium (DMEM) with 10% (v/v) fetal bovine serum (FBS, Corning) and maintained in growth medium supplemented with selective antibiotics following the manufacturer's instructions. RAW264.7 cells (ATCC) were cultured in high-glucose DMEM with 10% (v/v) FBS and then were subcultured by detachment with a cell scraper. C3H10T1/2 cells (ATCC) were cultured in high-glucose DMEM with 10% (v/v) FBS and 1× penicillin/streptomycin (Thermo Fisher). 3T3-L1 cells (ATCC) were cultivated in high-glucose DMEM with 10% (v/v) calf serum and 1× penicillin/streptomycin to maintain the undifferentiated status. At two days post-confluence, 3T3-L1 preadipocytes were induced to differentiate into adipocytes using an adipogenic cocktail containing 10 μg/mL insulin, 1 μM dexamethasone, and 0.5 mM 3-isobutyl-1-methylxanthine in 10% FBS DMEM. Two days post-induction, the cell medium was switched to the maintenance medium of 10% FBS DMEM supplemented with 2.5 μg/mL insulin. HSA-PG3 or P-G3 treatments (10 μg/mL) of 3T3-L1 cells were from differentiation Day 0 to Day 6.

### 3.3. HEK Reporter Cell Treatment and the SEAP Assay

HEK-Blue TLR3 cells (5×10⁴ cells/well) were seeded and cultured overnight in 96-well plates in DMEM and then stimulated with murine plasma or liver total RNA. After 48 h, the activation of reporter cells was determined with a Quanti-blue medium (Invivogen). For the respective experiments, TLR inhibitor (TLR3/dsRNA complex inhibitor or TLR8 inhibitor CU-CPT9a from Sigma-Aldrich) or cationic P-G3 was added 30 min before the stimulus. In the group with ribonuclease I (RNase I, Thermo Fisher Scientific), plasma or RNA was digested with 5 U of enzyme in a water bath at 37° C. for 60 min. For other TLR reporter lines, different seeding densities were adopted from the product manuals, while the other conditions were the same as in HEK-Blue TLR3 cells.

### 3.4. RAW264.7 Cell Treatment

RAW264.7 cells at 1×10⁵/cm² were seeded overnight in 12-well plates in 1 mL of DMEM containing 1% (v/v) FBS. Cells were treated with 20 μg/mL murine liver RNA; the scavenging group was pretreated with 5 μg/mL P-G3 for 30 min. After 24 h of treatment, the culture medium was harvested to measure the secreted TNF-α.

### 3.5. ELISA

Secreted TNF-α was determined with an uncoated ELISA kit (Thermo Fisher Scientific). In brief, RAW264.7 cell culture medium was centrifuged at 5,000×g for 10 min, and the supernatant was taken for the assay. A Nunc MaxiSorp 96-well plate was coated with 100 μL/well TNF-α antibody overnight at 4° C. and then blocked with 200 μL of ELISA/ELISPOT diluent at room temperature (RT) for 1 h. Then, 100 μL of each standard and sample was incubated at 4° C. overnight. The diluted detection antibody, streptavidin-TRP, and TMB solution were added sequentially according to the manufacturer's manual. Last, 2 N H₂SO₄ was added to stop the reaction, and the plate was read at 450 nm with a subtraction of readings at 570 nm for calculating the concentration.

### 3.6. HSA-PG3 Generation and Characterization

Materials and reagents: P-G3 was purchased from Dendritech, Inc. (USA). Human serum albumin (HSA) was purchased from Sigma-Aldrich (USA). Cy5-NHS was purchased from Fisher Scientific (USA). Ultrapure water (18.2 MΩ) was obtained from a Milli-Q water purification system.

Preparation of the HSA-PG3 complex: A methanol solution of 10 mg of P-G3 was vacuum dried, dissolved in 1 mL of water, and lyophilized overnight to remove any remaining methanol. Then, with sonication, the P-G3 solution (10 mg of P-G3 in 1 mL of H₂O) was added dropwise into the HSA solution (100 mg of HSA in 9 mL of H₂O). The mixture was sonicated for another 10 min to obtain the HSA-PG3 complex with an HSA:P-G3 ratio of 10:1. Complexes with different HSA:P-G3 ratios were prepared in a similar way. The Cy5-labeled HSA-PG3 complex was prepared by adding Cy5-labeled P-G3 (Cy5-NHS:P-G3 in a mass ratio of 1:50) into an HSA water solution. The complexes' hydrodynamic diameter, zeta potential, count rate, polydispersity index, and stability were measured using a Malvern Nano ZS90 Zetasizer. The morphology was determined using an FEI Titan Themis 200 TEM. The release curve was measured using the Cy5-labeled HSA-PG3 complex. Briefly, 1 mL of the complex (1 mg/mL) sealed in a cellulose dialysis bag (Spectra/Por 2, MWCO 12,000-14,000, Spectrum, USA) was immersed in 9 mL of PBS in a centrifuge tube. The tube was shaken on a shaking bed at 200 rpm at 37° C. At different time intervals, the dialysis bag was transferred to a new centrifuge tube with fresh PBS. The fluorescence (Ex/Em: 620/670 nm) of the solution in tubes was measured using a FLUOstar Optima FL microplate reader.

HSA-PG3 characterization—DNA-binding assay: The DNA-binding ability of the complexes was assessed using the Quant-iT PicoGreen DNA Assay Kit (Fisher Scientific, USA). PicoGreen solutions were diluted 2,000-fold in TE buffer (Fisher Scientific, USA). Salmon sperm DNA (Fisher Scientific, USA) was added as a standard DNA with a final concentration of 2 μg/mL. The mixture was incubated at room temperature for 30 min. Samples were added to a 96-well black plate (50 L/well). Then, the mixture of PicoGreen and standard DNA (50 L/well) was added to the same plate. After shaking for 15 min, fluorescence (Ex/Em: 490/520 nm) was measured by a FLUOstar Optima FL microplate reader.

HSA-PG3 characterization—RNA-binding assay: The RNA-binding ability of the complexes was assessed using the Quant-iT RNA assay kit (Thermo Fisher Scientific). Detecting reagents were diluted 1,000-fold in TE buffer (Fisher Scientific, USA). E. coli rRNA standard was added with a final concentration of 2 μg/mL. The mixture was incubated at room temperature for 10 min. Samples were added to a 96-well black plate (10 μL/well). Then, the mixture of detecting reagent and standard RNA (200 L/well) was added to the same plate. After shaking for 15 min, fluorescence (Ex/Em: 620/670 nm) was measured by a FLUOstar Optima FL microplate reader.

HSA-PG3 characterization—Poly (I:C) scavenging assay: HEK-Blue™ TLR3 cells were seeded and cultured as described above in Section 2.3. P-G3 or HSA-PG3 (1 μg/mL, counting P-G3 in complexed HSA-PG3 unless otherwise stated) was used to treat cells for 30 min before adding 2 μg/mL poly (I:C). After 24 h, the activation of the reporter cells was determined with a Quanti-blue™ medium.

HSA-PG3 characterization—Cytotoxicity assay: The cytotoxicity of the complexes was measured by the Cell Counting Kit-8 assay (CCK-8, Dojindo, USA). 3T3-L1 cells were seeded in a 96-well plate at 1×10⁴ cells/well, kept quiescent overnight, and cultured for another 48 h after adding HSA-PG3 or P-G3. CCK-8 solution (10% in media) was added to each well, and the cells were incubated at 37° C. for 1 h. The absorbance was measured at 450 nm with a FLUOstar Optima FL microplate reader.

### 3.7. Cy5-Labeled P-G3 or HSA-PG3 In Vivo Distribution Imaging

Chow-fed mice were fed a high-fat diet (HFD, 60% kcal from fat, Research Diets) for one week to reduce the possible autofluorescence signal from the chow diet. 200 g Cy5-labeled HSA-PG3 or P-G3 was administered to mice via the intraperitoneal (IP) route. At Day 1, Day 8, and Day 15 after the IP injection, the mice were sacrificed, and the tissues were imaged by using a PerkinElmer IVIS system. In addition, 200 μg or 50 μg of Cy5-labeled HSA-PG3 was given to mice via the intravenous (IV) route or locally into subcutaneous inguinal WAT, respectively. At Day 3 and Day 7 after the injection, the mice were sacrificed, and the fluorescence signal in the tissues was analyzed using the aforementioned system.

### 3.8. Animal Studies

The animal protocol is reviewed and approved by the Institutional Animal Care and Use Committee (IACUC) of Columbia University. For the treatment of DIO mice with P-G3, 8-week-old male C57BL/6 mice were fed a HFD for 20 weeks to induce obesity and then were given P-G3 (10 μg/g BW) intraperitoneally twice weekly while kept on HFD feeding. For HSA-PG3 used in the obesity prevention study, 8-week-old male mice were fed a HFD and administered HSA-PG3 (10 μg/g BW) intraperitoneally twice weekly. Body weight was monitored weekly. For the glucose tolerance test (GTT), the mice received 20% glucose injection (2 mg/g BW) by IP injection after 16 h of fasting. For the insulin tolerance test (ITT), mice were IP injected with insulin (0.75 U/kg BW) after 4 h of fasting. The blood glucose levels were measured by using a OneTouch glucometer at the indicated time points via tail vein bleeding. For indirect calorimetric analyses, mice were single-housed in metabolic cages (Comprehensive Lab Animal Monitoring System) to monitor the food intake, activity, heat production, O₂ consumption, and respiratory exchange ratio during the 24-h dark/light cycle for 5 days. After 16 h of fasting and 4 h of refeeding, the mice were sacrificed by CO₂ asphyxiation.

For HSA-PG3 local treatment of subcutaneous fat, 26-week-old male mice were treated with HSA-PG3 or HSA once weekly by subcutaneous injection into the inguinal fat while on HFD feeding. For each injection, 50 g of HSA-PG3 (10:1 ratio) or 500 g HSA (equivalent to 50 g of HSA-PG3) was diluted in 300 μL of PBS solution and injected into three spots on one side of the inguinal fat. The vehicle control (PBS solution) was administered to the other side of the subcutaneous fat. Mice were sacrificed at five weeks post-injection.

### 3.9. Gene Expression Analysis

Tissues or cells were lysed into TRIzol™ reagent (Thermo Fisher Scientific), followed by the addition of chloroform for phase separation, and then were purified using a Tri-Isolate RNA Pure Kit (IBI Scientific). 1 μg of RNA was used to synthesize cDNA with a High-Capacity cDNA Reverse Transcription kit from Applied Biosystems. A Bio-Rad CFX96 Real-Time PCR system was used to perform quantitative real-time PCR (Q-PCR) with GoTaq qPCR Master Mix (Promega). The relative gene expression was calculated by using the ΔΔCt method, and Rpl23 was used as a reference gene.

### 3.10. Western Blotting

Total protein from eWAT was extracted in the IntactProtein Lysis Buffer (GenuIn Biotech #415) and analyzed using the same protocol as described in a previous study [35]. The antibodies used in this study were FASN (CST #3180), C/EBPα (Santa Cruz, sc-61), PPARγ (CST #2443), and GAPDH (Proteintech, #HRP-60004).

### 3.11. Histology and Immunohistochemistry

After dissection, tissues were immediately fixed in a 10% formalin buffered solution. Two days after fixation, tissues were embedded in paraffin, sectioned, stained with hematoxylin and eosin (H & E), and photographed under a microscope (Olympus IX71). The images were processed using ImageJ software. Frozen eWAT sections were used for the immunohistochemical staining. eWAT sections were incubated with Caveolin-1 (D46G3) (CST, #3267) at a 1:250 dilution overnight, and then anti-rabbit Alexa 488 antibody (Thermo Fisher Scientific, #A27034) was used at a 1:1000 dilution.

### 3.12. Statistical Analysis

The data are presented as the mean±SEM. The significance between groups was evaluated using ANOVA for multiple comparisons or t-test for comparisons between two groups. A paired t-test was used to assess the significance between the subcutaneous fat on the HSA-PG3 treatment side and that on the nontreated side. The significance level was set to a p value<0.05.

### 4. Examples and Results

### 4.1. Plasma Cell-Free RNA is Increased in Obese Mice and Activates TLR3

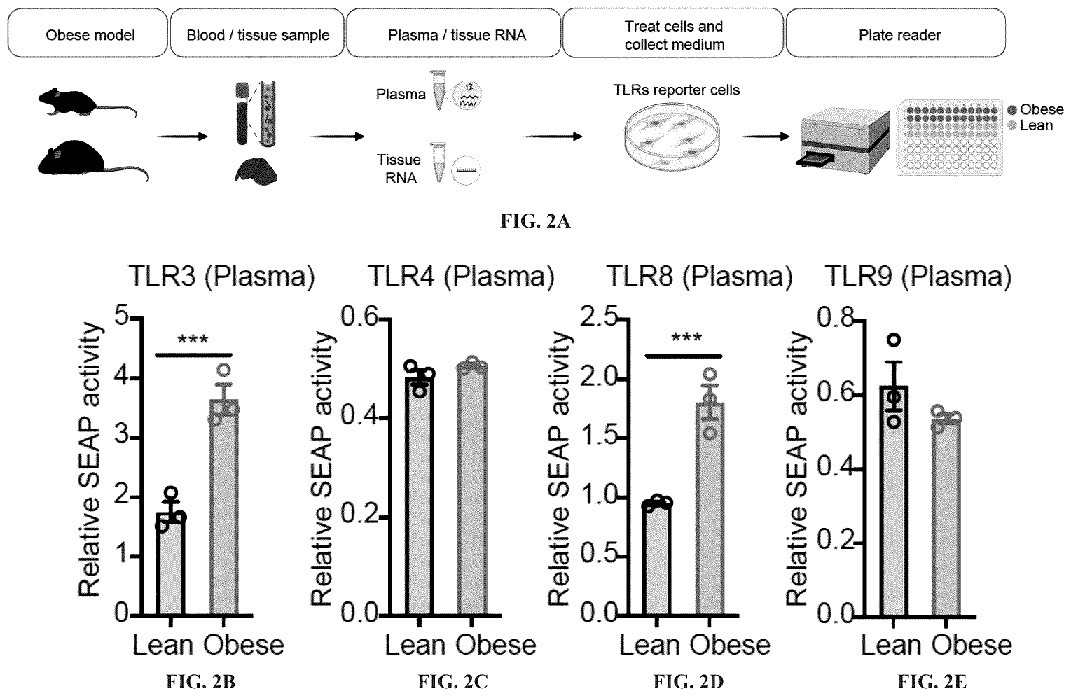

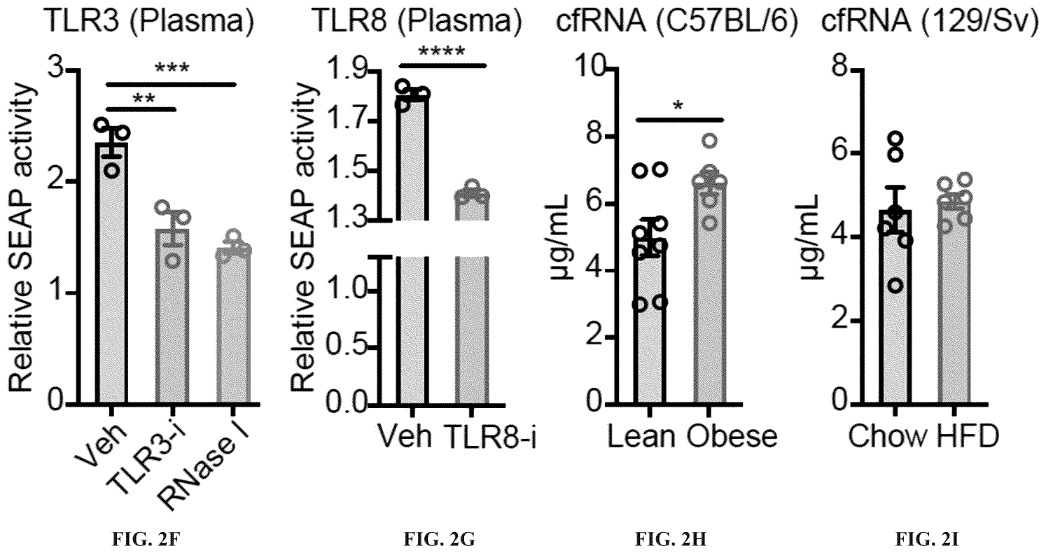

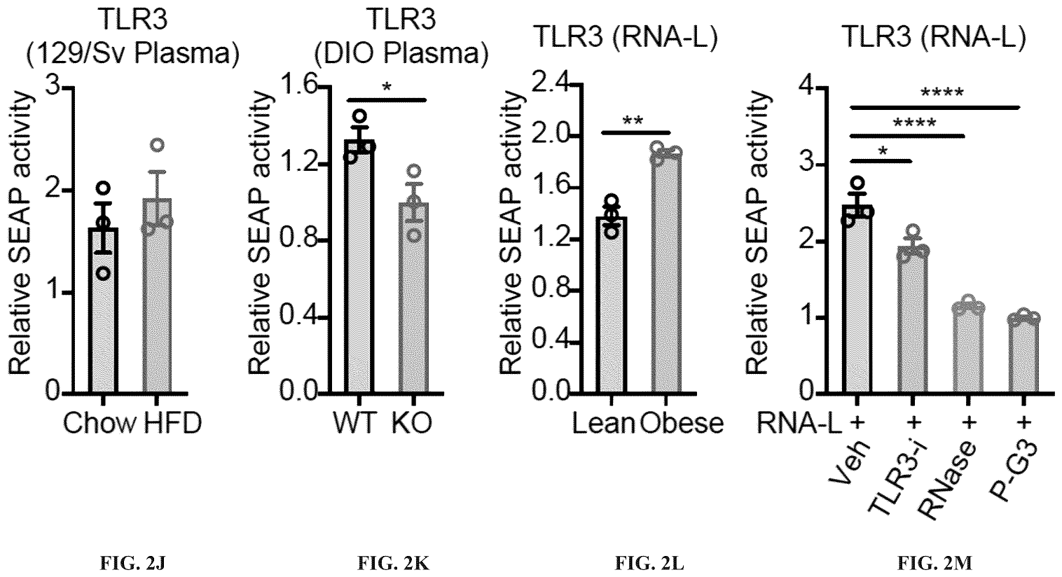

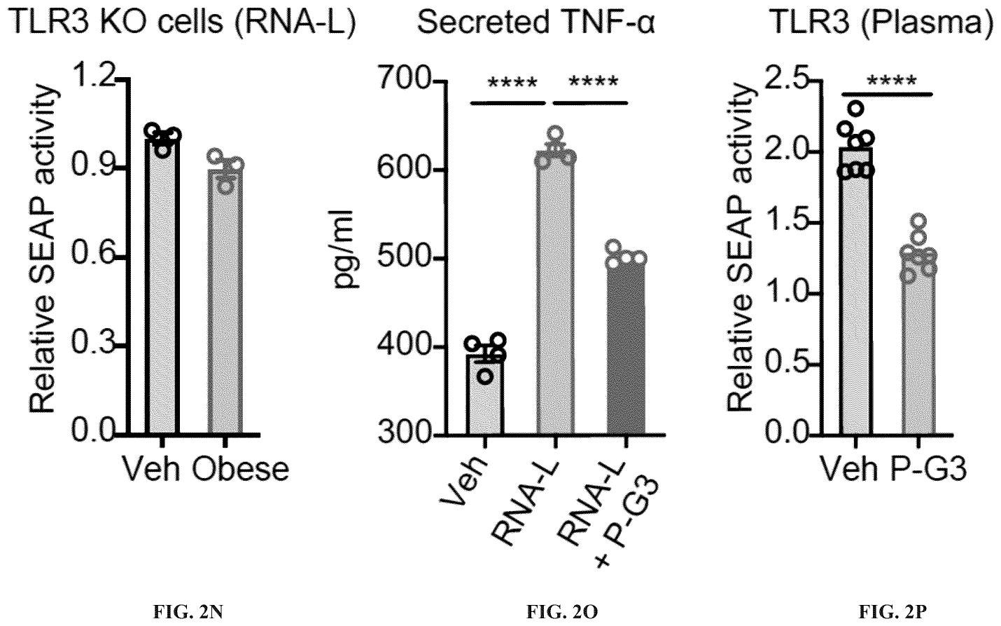

To better understand the pathogenesis of chronic inflammation in obesity, which involves multiple organs, we investigated whether proinflammatory factors were released into the plasma during obesity to mediate interorgan communication. Because TLRs play a prominent role in activating the innate immune response, we compared the activation of TLRs by the plasma of obese mice to lean mouse controls (FIG. 2A). Interestingly, plasma from diet-induced obese (DIO) mice activated TLR3 and TLR8 but not TLR4 and TLR9 (FIGS. 2B-2E). The activation of TLR3 and TLR8 can be blocked by specific TLR3 and TLR8 inhibitors (FIGS. 2F, 2G), reinforcing the presence of their ligands in the obese plasma. Treating the obese plasma with RNase I to deplete single-strand RNA (ssRNA) surprisingly blocked the activation of TLR3 (FIG. 2F), whose classic ligand is viral double-stranded RNA (dsRNA), suggesting that cell-free ssRNAs exist in the plasma and may form double-stranded structures to activate TLR3 [36]. Indeed, the cell-free RNA (cfRNA) concentration in the obese plasma was increased by ˜40% (FIG. 2H). Next, an investigation into whether the dietary difference accounted for the surge in plasma cfRNA was also conducted.

In the obesity-resistant 129/Sv mice, HFD feeding failed to increase the cfRNA concentration (FIG. 2I) and consequent TLR3 activation (FIG. 2J). To further discount dietary effects, we implemented the genetic model of PPARα KO mice, which gain less weight when fed a HFD compared to that of wild-type (WT) littermate controls [37, 38]. Again, the KO plasma showed less activation of TLR3 reporter (FIG. 2K). Therefore, obesity per se rather than the diet likely increases circulating cfRNA to activate TLR3.

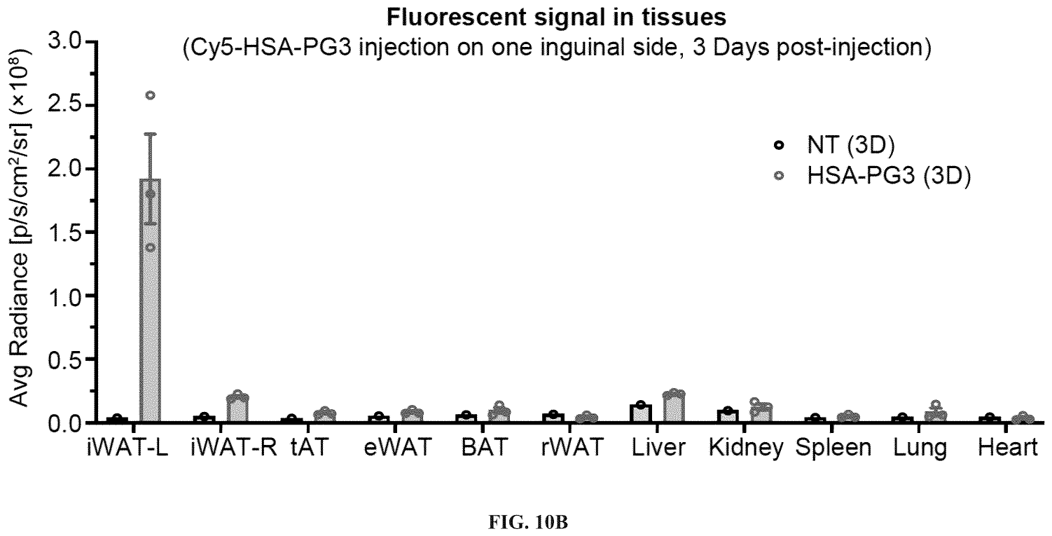

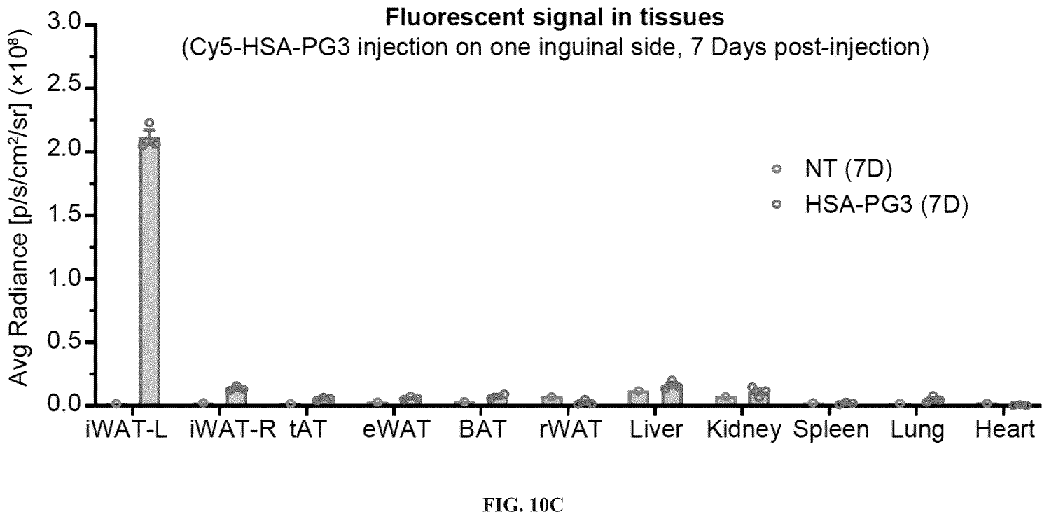

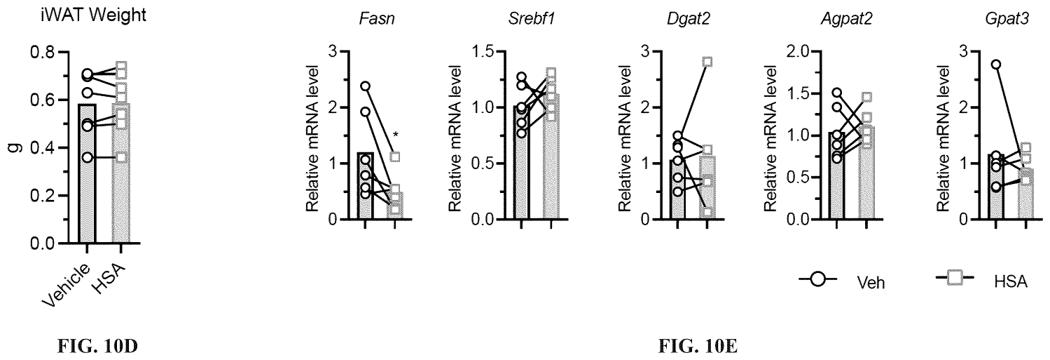

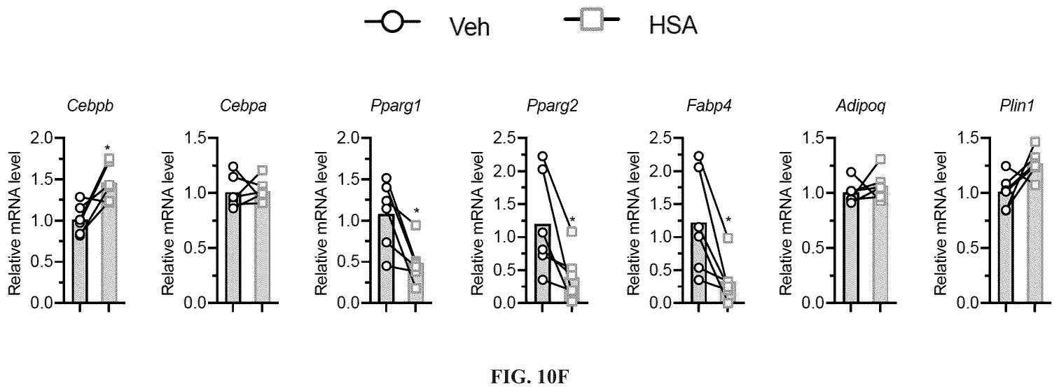

To exclude the interference of other plasma components in activating TLR3, we purified total RNA from the liver (RNA-L), one of the major metabolic organs affected in obesity. Interestingly, the RNA isolated from obese liver displayed stronger activation of TLR3 than that of the lean mouse liver RNA (FIG. 2L). This activation of TLR3 was suppressed by digesting the RNA, blocking the receptor, or ablating the receptor (FIGS. 2M and 2N), confirming the specificity of TLR3. From a functional perspective, RNA isolated from obese mouse livers induced the secretion of TNF-α from murine macrophage RAW264.7 cells (FIG. 10). In agreement with the scavenging function of cationic P-G3, the activation of TLR3 by liver RNA and plasma from obese mice was largely inhibited by P-G3 (FIGS. 2M, 2P). Additionally, P-G3 suppressed the liver RNA-augmented secretion of TNF-α from macrophages (FIG. 2O). Together, these results imply positive correlations among obesity, cfRNA, and TLR3 activation, highlighting the potential of P-G3 application to ameliorate chronic inflammation in obesity.

4.2. Metabolic Improvements in Diet-Induced Obesity from P-G3 Treatment

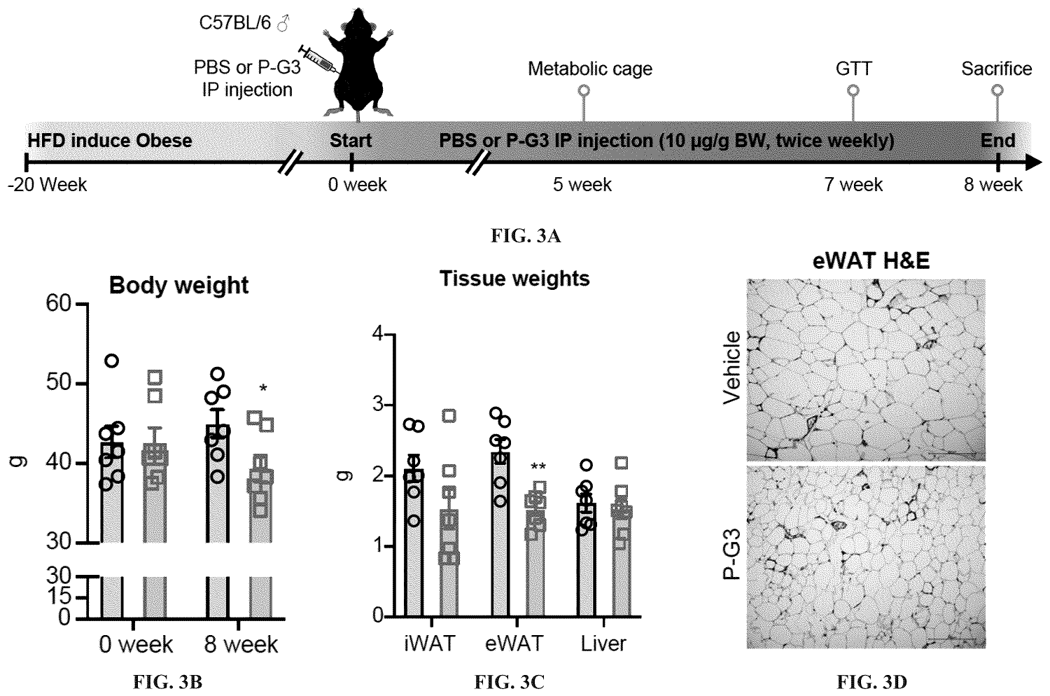

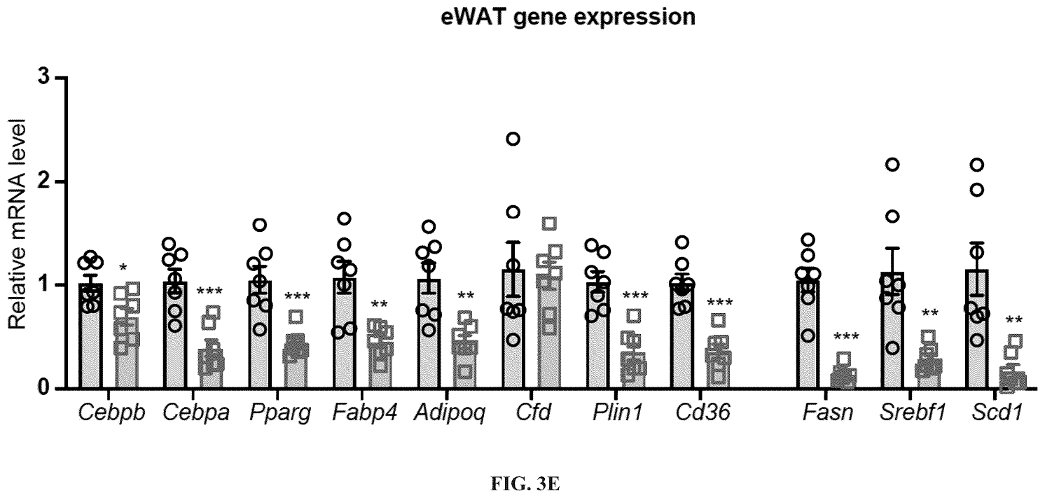

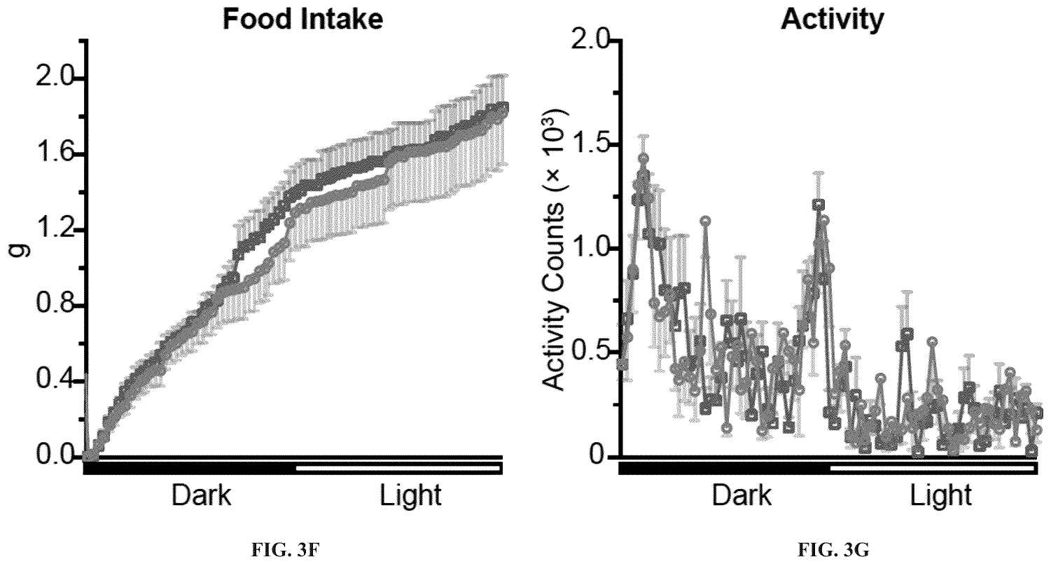

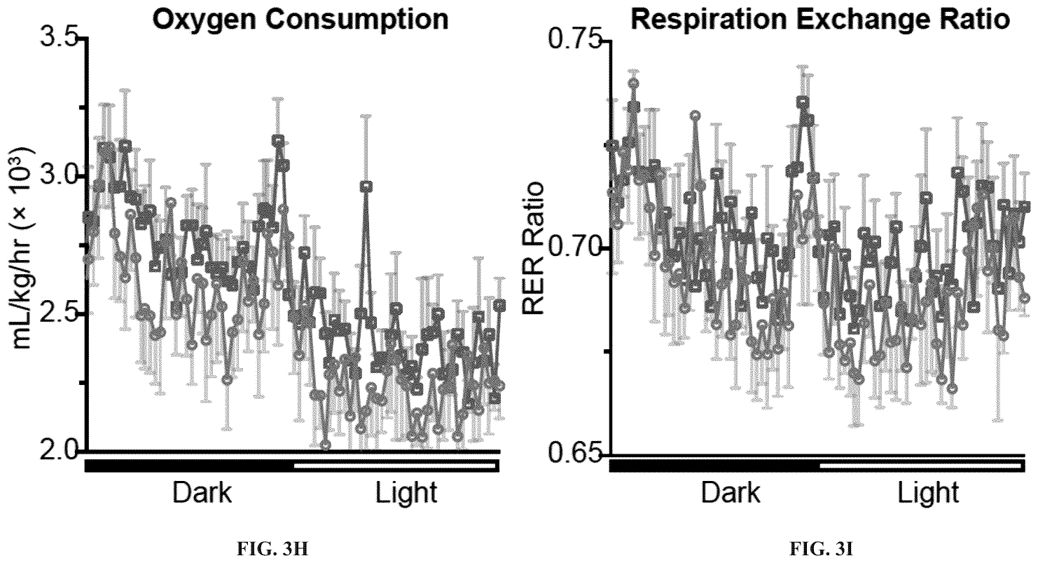

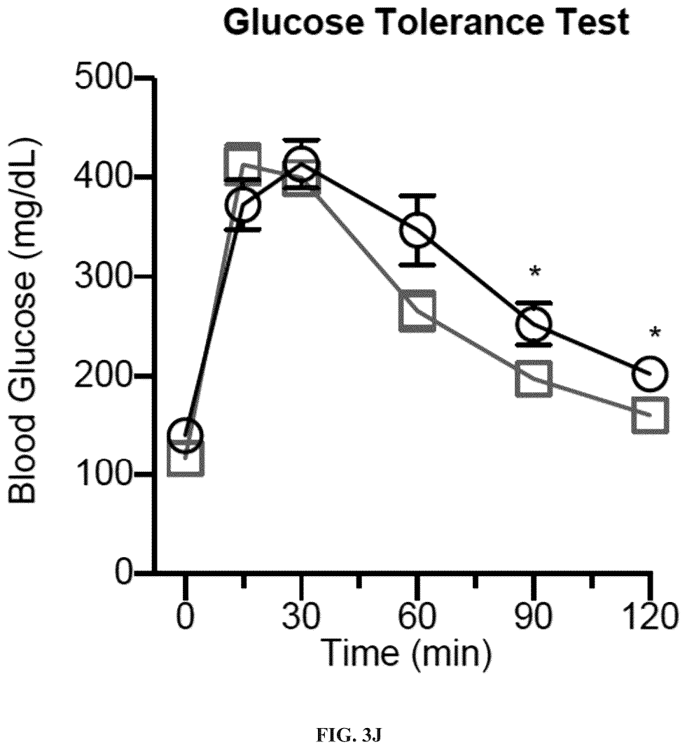

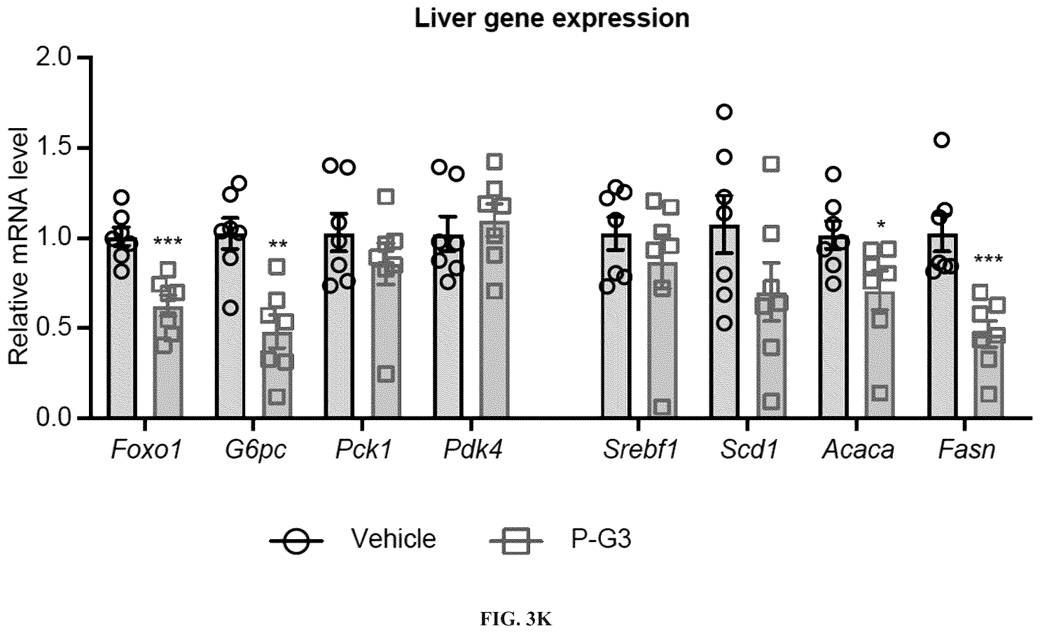

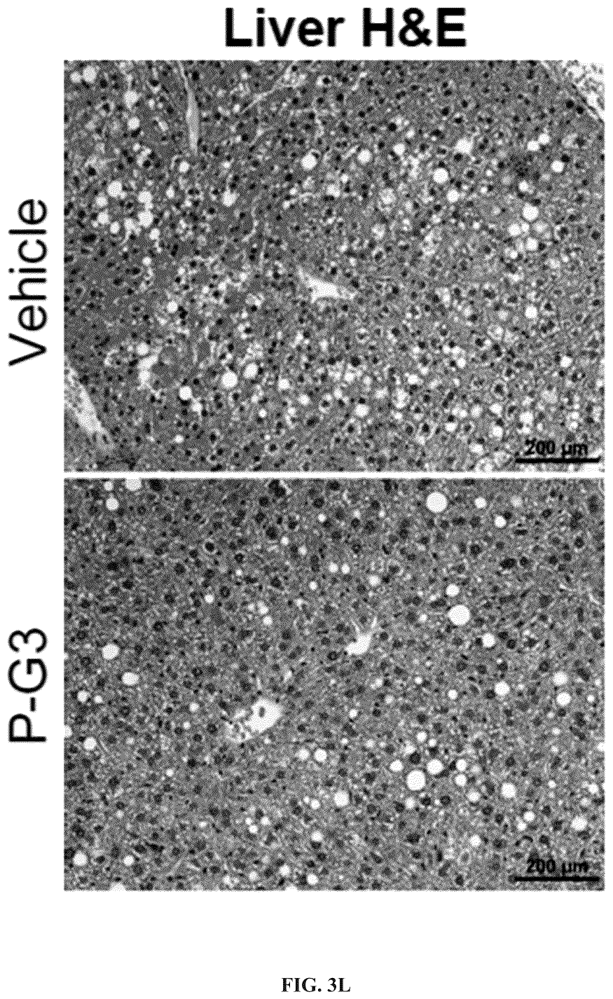

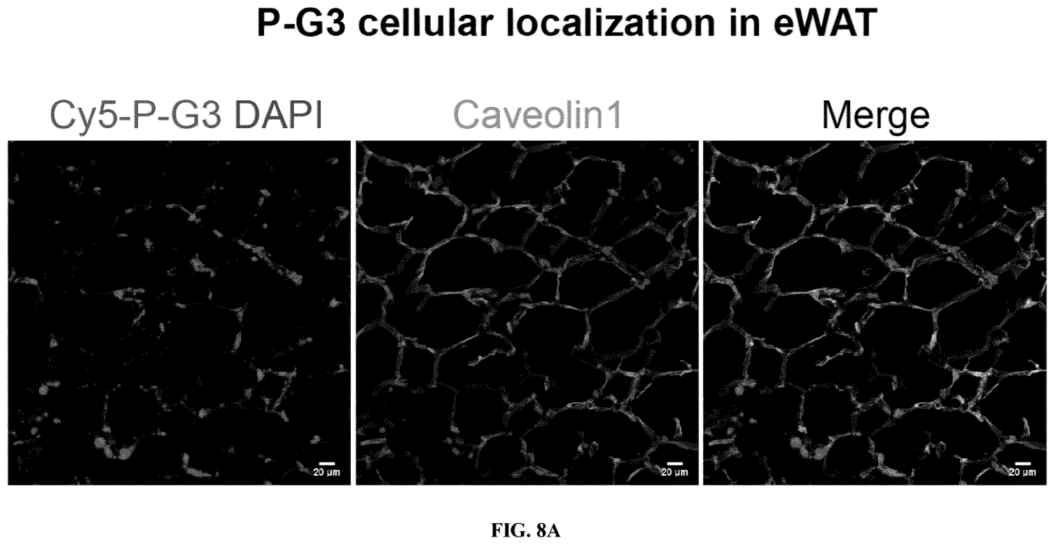

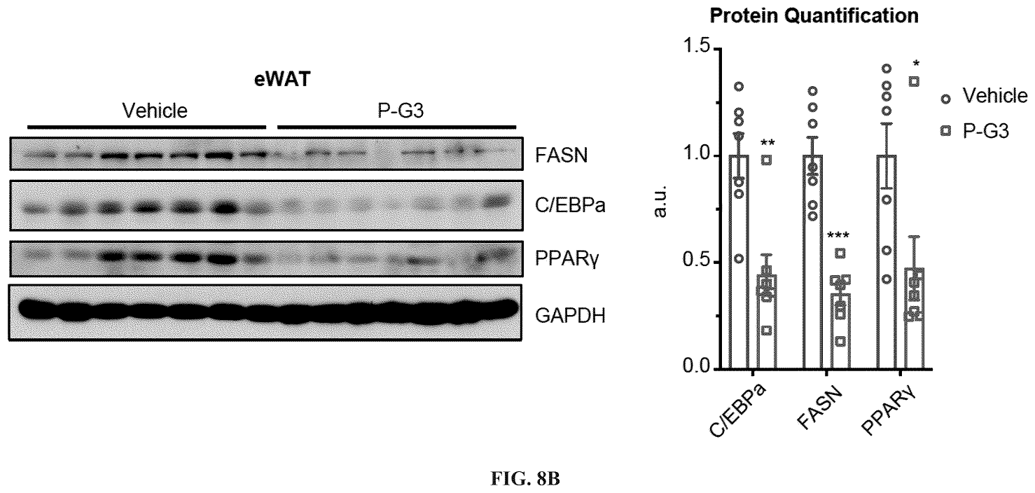

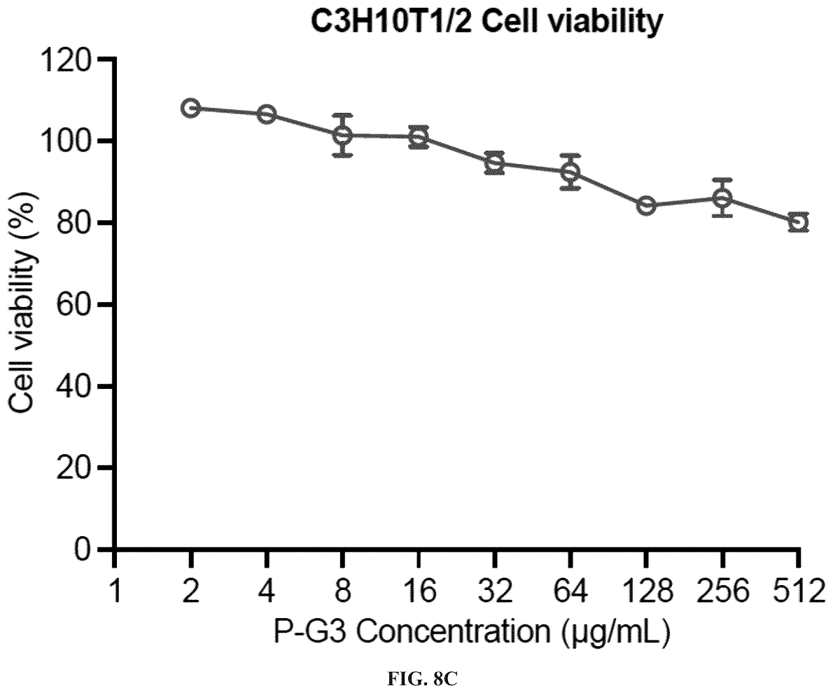

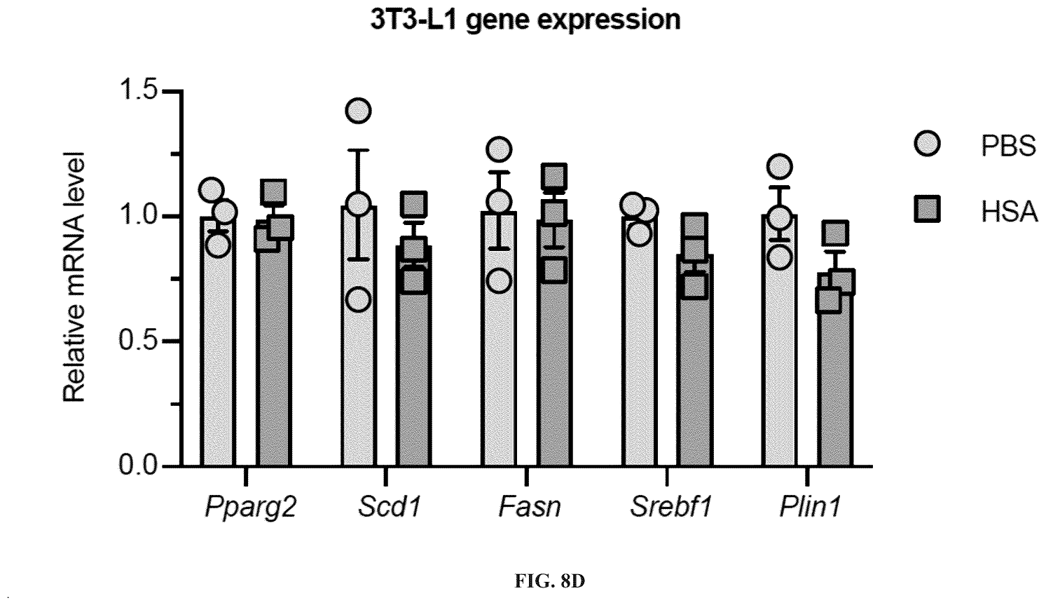

Next, we directly tested the metabolic effects of P-G3 treatment in obese mice. Male C57BL/6 mice were fed a HFD for 20 weeks to fully establish obesity. Then, P-G3 treatment was started via the regular IP delivery route (FIG. 3A). An eight-week treatment of P-G3 resulted in a significant anti-obesity effect (FIG. 3B), with a stronger inhibition of epididymal WAT (eWAT) mass than subcutaneous inguinal WAT (iWAT) (FIG. 3C) and no effect on liver mass. In line with the decreased fat mass, adipocytes in the eWAT of P-G3-treated mice were smaller overall (FIG. 3D), accompanied by the downregulation of prominent adipocyte regulators (Cebpb, Cebpa, and Pparg) and their downstream target genes (Fabp4, Adipoq, Plin1, and Cd36) (FIG. 3E). Interestingly, immunofluorescent staining showed even distribution of P-G3 signal into eWAT with overlapping with adipocyte marker Caveolin-1 (FIG. 8A), suggesting P-G3 enters adipocytes to function. In addition, key lipogenesis genes (Fasn, Srebf1, and Scd1) were all repressed by P-G3 (FIG. 3E). The decrease in the key regulators PPARγ, C/EBPα, and FASN was verified at the protein level (FIG. 8B). These data indicate an anti-obesity effect of P-G3.

To understand the anti-obesity effect of P-G3 using mice, we conducted metabolic cage analysis to assess energy balance. Without a reduction in food intake or an increase in locomotion activity, P-G3 treatment modestly increased oxygen (O₂) consumption (FIG. 3F-3H), indicating a higher metabolic rate. The unchanged food intake and locomotion activity also suggest that P-G3 did not cause sickness in mice at our dose of 10 μg/g BW. Indeed, P-G3 demonstrated low cytotoxicity in two of the most widely used adipocyte progenitor cell lines, with >90% cell viability at a concentration of 64 μg/mL (FIG. 8C), which is approximately 6-fold greater than the in vivo dose. Furthermore, P-G3-treated mice showed a higher respiratory exchange ratio (RER) (FIG. 3I), which is an indicator of fuel preference between fatty acids and carbohydrates. This higher RER corresponds to higher carbohydrate utilization, counteracting insulin resistance in obesity. Consistently, the obesity-induced impairment of glucose tolerance was rectified by P-G3 treatment (FIG. 3J). Moreover, obesity is associated with dysregulated glucose and lipid metabolism in the liver, whereas P-G3 treatment significantly repressed the key genes involved in hepatic glucose production (G6pc and Foxo1) and, concurrently, the key genes involved in lipogenesis (Fasn and Acaca) (FIG. 2K), in line with a mild alleviation of hepatic steatosis (FIG. 3L) as well as the improved glucose homeostasis. Collectively, P-G3 treatment shows systemic metabolic improvements that may aid in combating obesity.

### 4.3. The Anti-Inflammatory Effect of P-G3 in Obesity

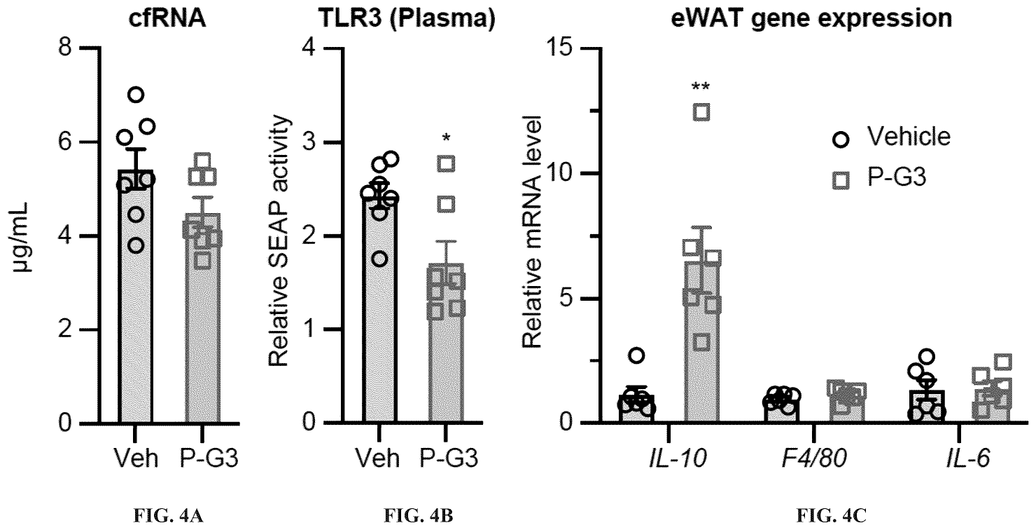

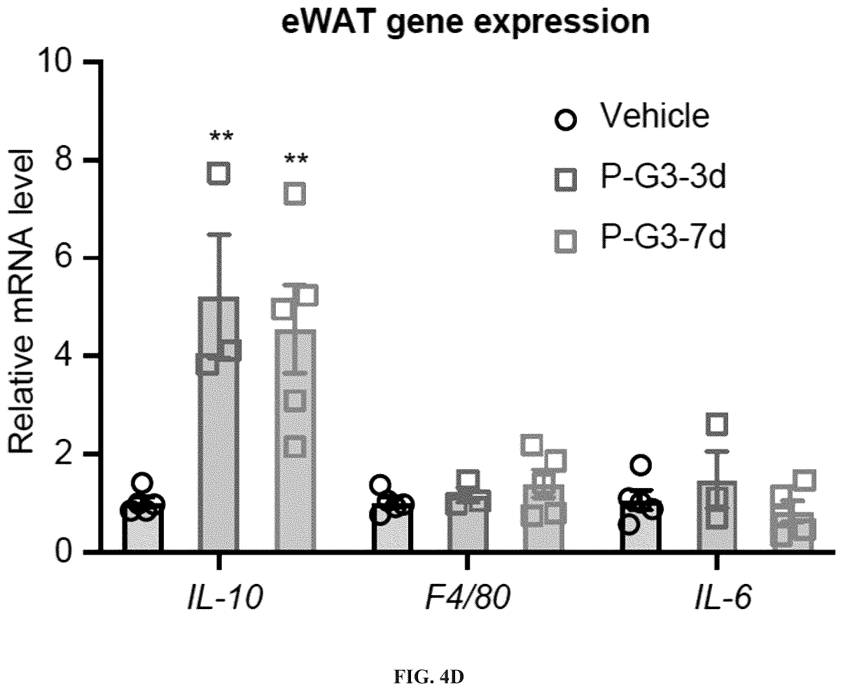

After 8 weeks of P-G3 treatment, the surge in plasma cfRNA levels in DIO mice tended to stabilize (FIG. 4A), accompanied by a significant decline in TLR3 activation potency (FIG. 4B). Given that WAT inflammation is a hallmark of obesity, we investigated the effect of P-G3 treatment on inflammation in eWAT. Interestingly, the anti-inflammatory gene IL-10 was drastically increased, while the proinflammatory markers IL-6 and F4/80 remained constant (FIG. 4C). Since the induction of IL-10 transcription might be secondary to improved adipose tissue health over chronic P-G3 treatment, we tested the direct effect of P-G3 in acute treatment. A single dose of P-G3 treatment significantly upregulated IL-10 after three days and was maintained after seven days. In contrast, IL-6 and F4/80 were unaffected (FIG. 4D). Therefore, P-G3 conveys an expected direct anti-inflammatory role in obesity.

4.4. Controlled Release of P-G3 Through the Formation of a Complex with Human Serum Albumin (HSA)

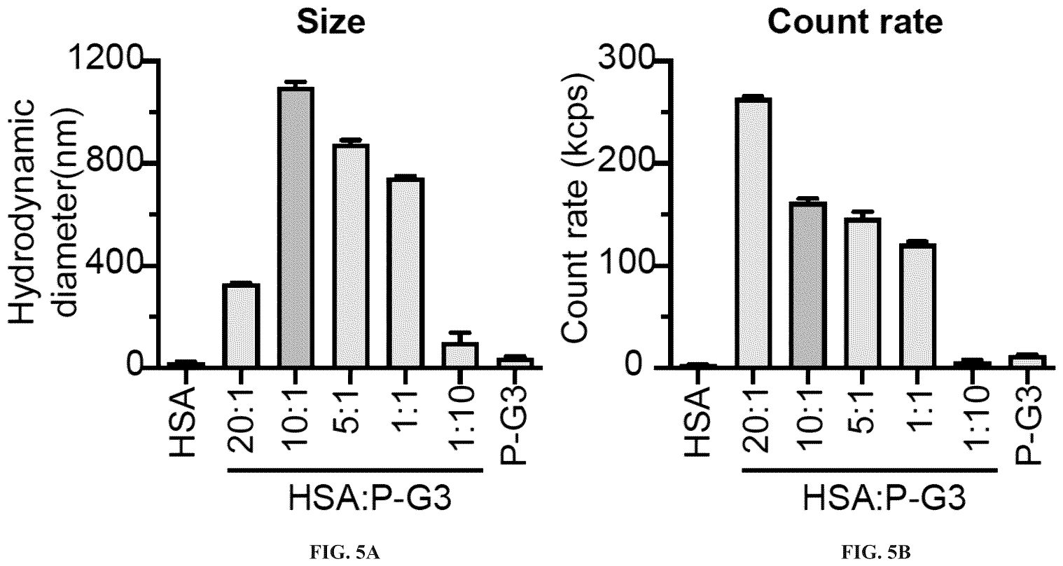

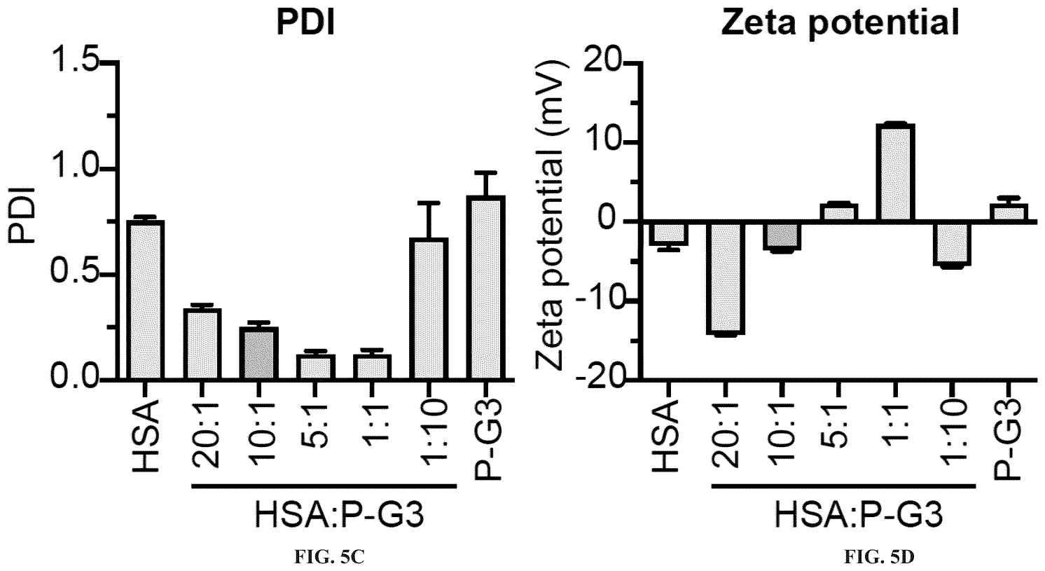

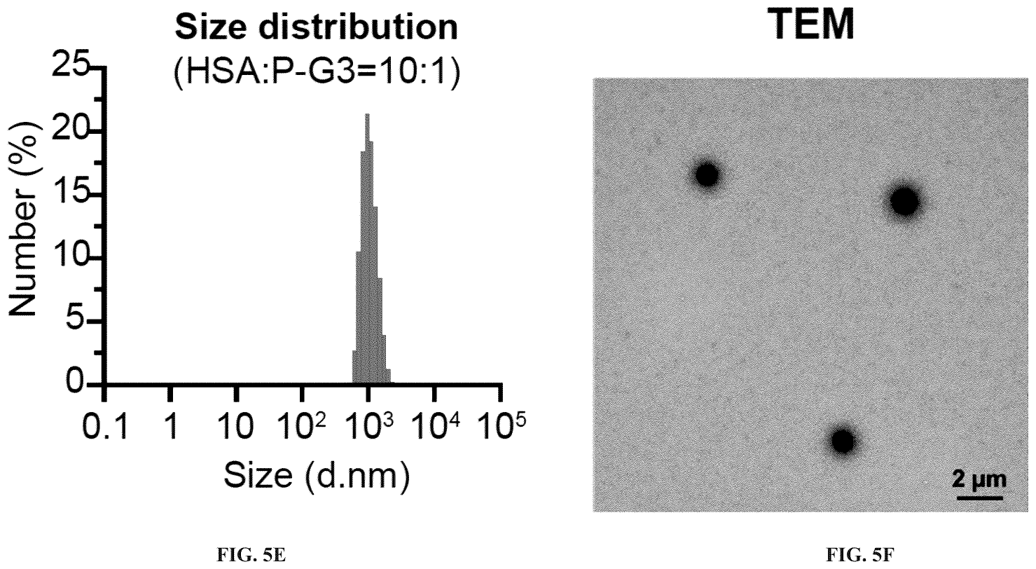

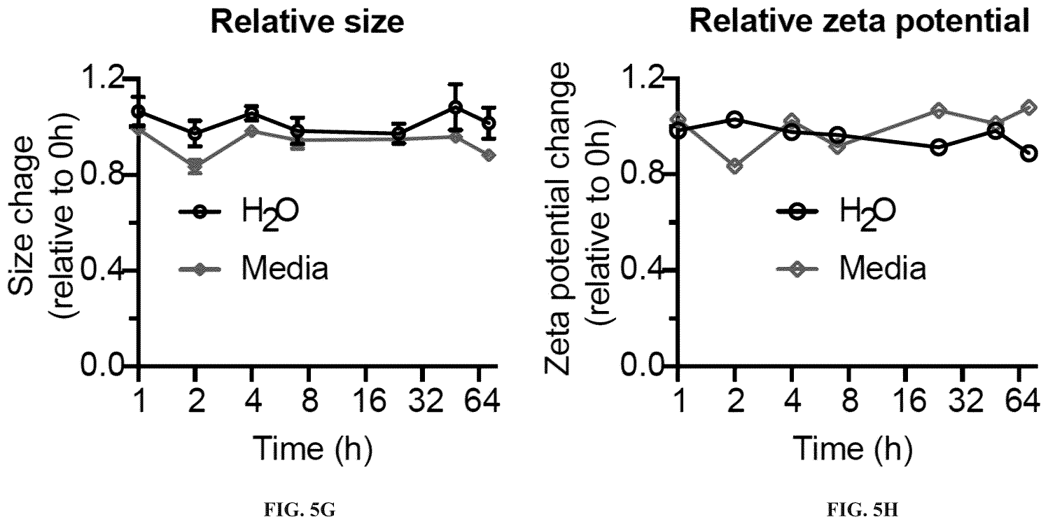

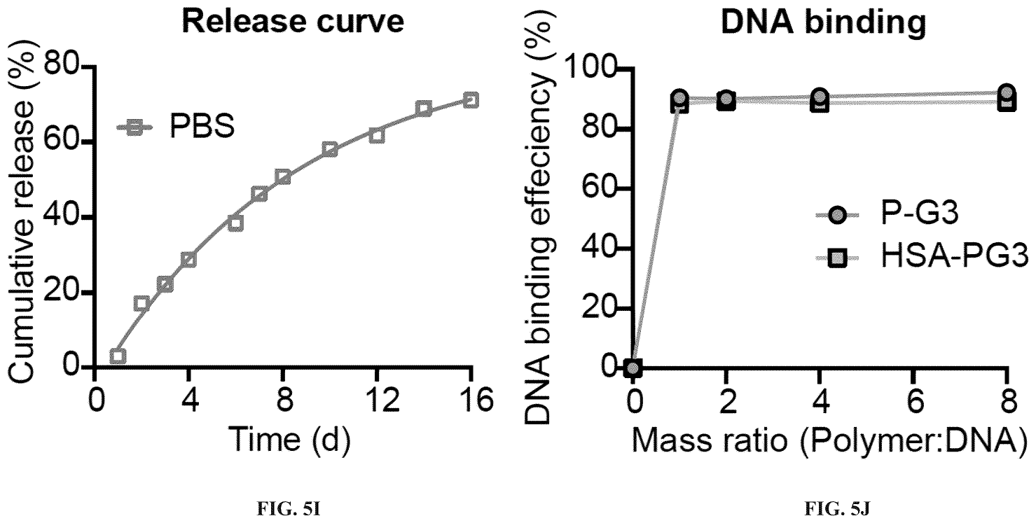

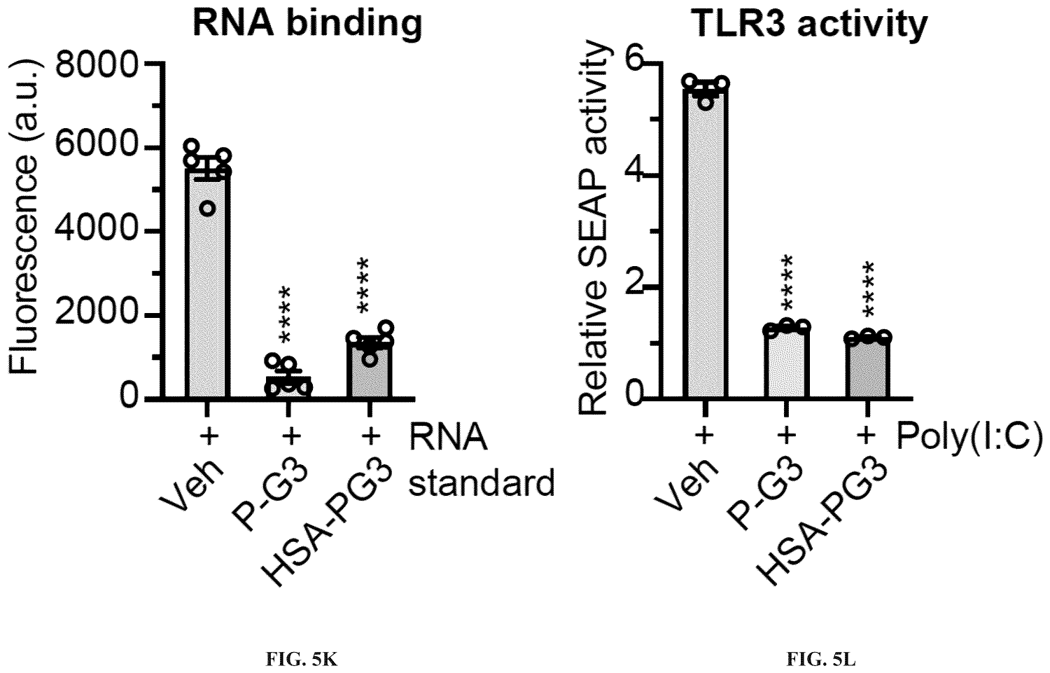

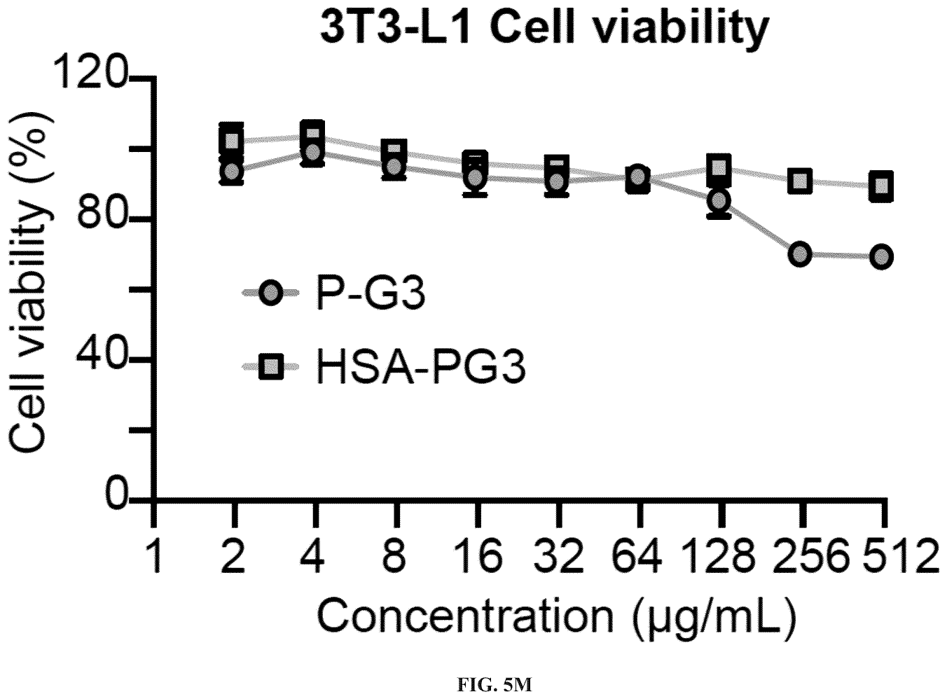

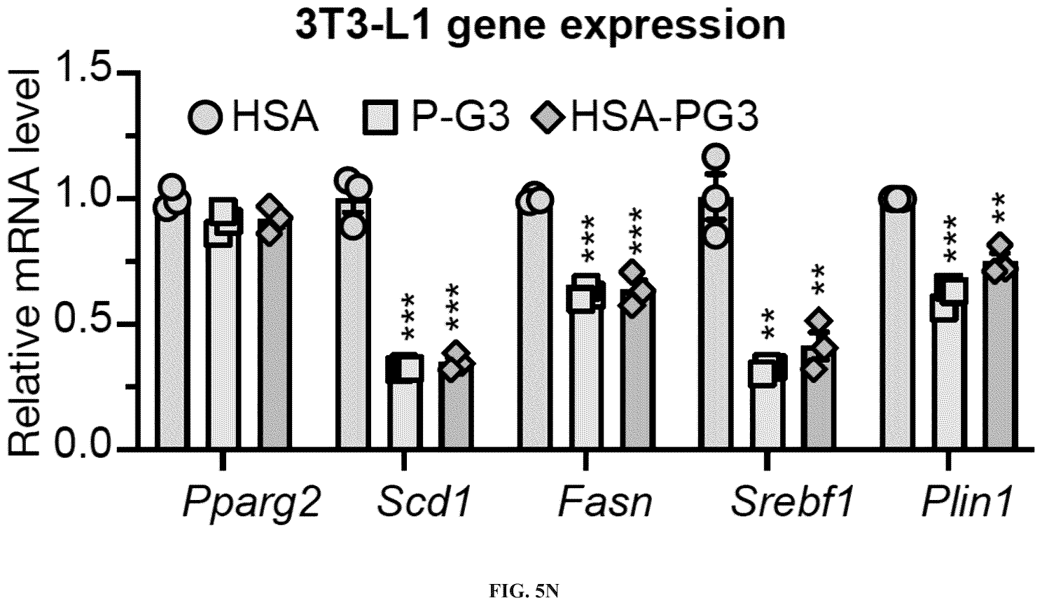

Despite the minimal toxicity of P-G3 observed in treating DIO mice, we endeavored to improve its translational applicability through controlled release for the sake of minimizing potential acute cationic toxicity. Albumin was chosen as a viable complexing component because it is abundantly present in the plasma and adipose tissue. Furthermore, it is widely used as a drug delivery carrier [39], such as in FDA-approved Abraxane, where HSA interacts with paclitaxel nanocrystals in a noncovalent manner to form an injectable formulation [40, 41]. We mixed HSA and P-G3 in different ratios and characterized their self-assembly behaviors, including particle size, count rate, polydispersity index (PDI), and zeta potential (FIGS. 5A-5D). Although both HSA and P-G3 are hydrophilic, dynamic light scattering (DLS) of their mixed solution showed that they contained particulates with a hydrodynamic diameter in the 100-1000 nm range and peaked at approximately 1 μm at an HSA:P-G3 mass ratio of 10:1 (FIG. 5A), indicating that they can self-assemble into nano- or microparticles. Hydrophilic molecules commonly show a low count rate and high PDI under DLS measurements, and their size and zeta potential are probably not the most accurate. Most importantly, the increase in the count rate and the decrease in the PDI validated the formation of uniform complexes of HSA-PG3 (FIGS. 5B, 5C). The formulation was finally optimized with a mass ratio of 10:1 between HSA and P-G3, resulting in nearly neutral spherical microparticles with a hydrodynamic diameter of 1098±36.2 nm and zeta potential of −3.55±0.25 mV (FIG. 4D-F).

HSA-PG3 complexes showed good stability in water or cell culture media (RPMI media with 5% FBS) for at least 3 days (FIGS. 5G, 5H). They likely associate with one another through charge-charge interactions. P-G3 was gradually released from the complexes within two weeks (FIG. 5I). HSA-PG3 complexes showed comparable ability in scavenging DNA and RNA as P-G3 (FIGS. 5J, 5K), resulting in the same potency in blocking TLR3 activation as a functional readout (FIG. 5L). Their comparable effect on cell viability at lower doses demonstrated a similar level of cytotoxicity, but HSA-PG3 showed higher cell viability at high doses of 250-500 μg/mL than P-G3 (FIG. 5M), suggesting its utility in local application. Moreover, HSA-PG3 and P-G3 had similar effects on inhibiting lipogenic genes in 3T3-L1 adipocytes (FIG. 5N), in contrast to the blunted effect of HSA (FIG. 8D). These characterizations highlight the successful complex formation of P-G3 with HSA, which may increase biocompatibility in treating obesity.

4.5. Treating Diet-Induced Obesity with HSA-PG3

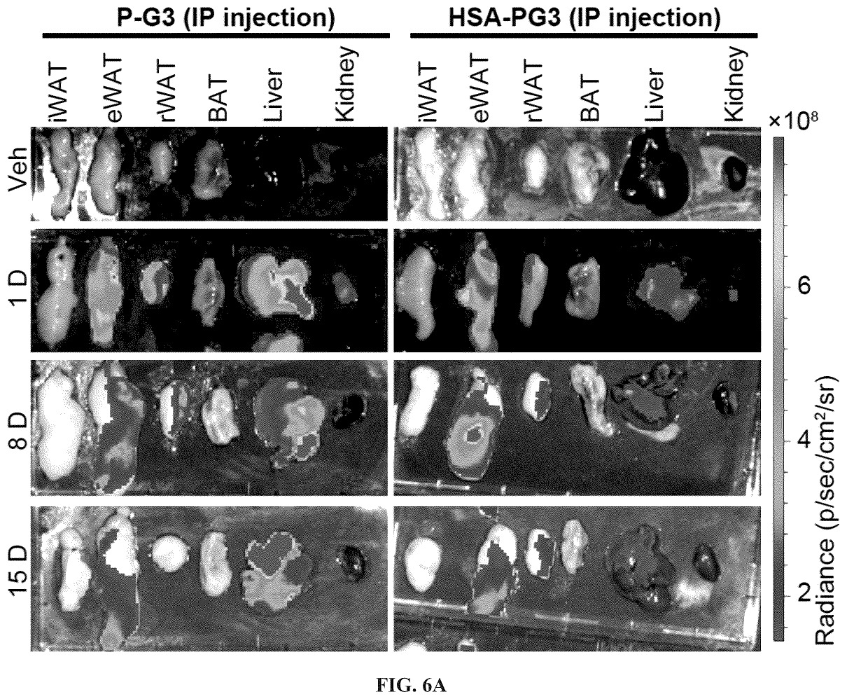

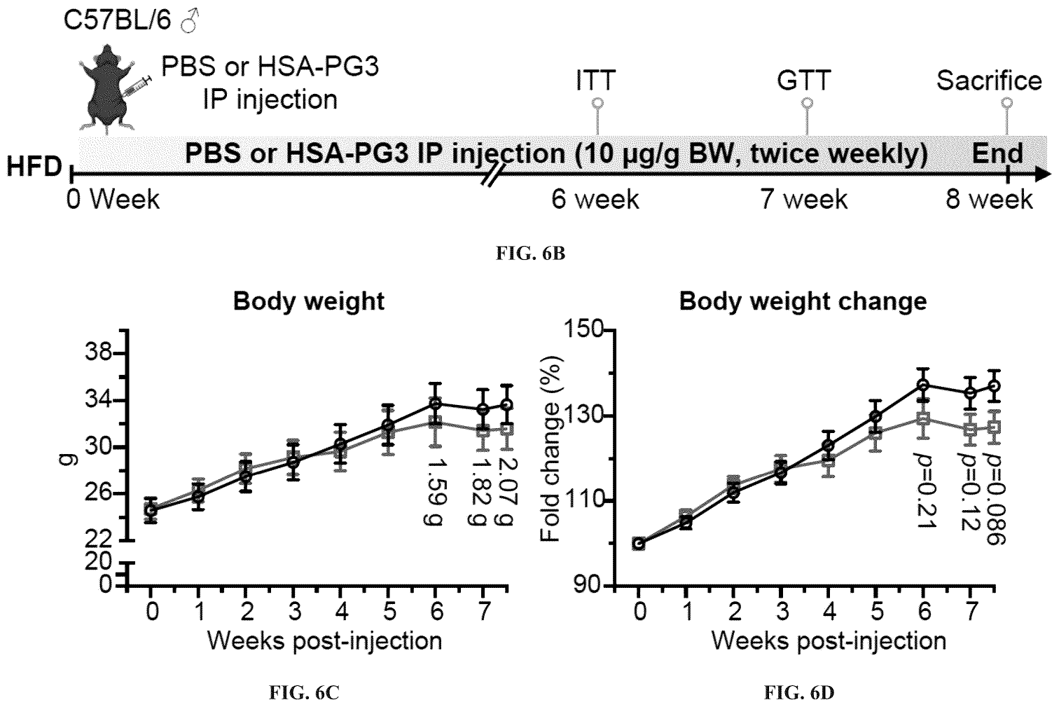

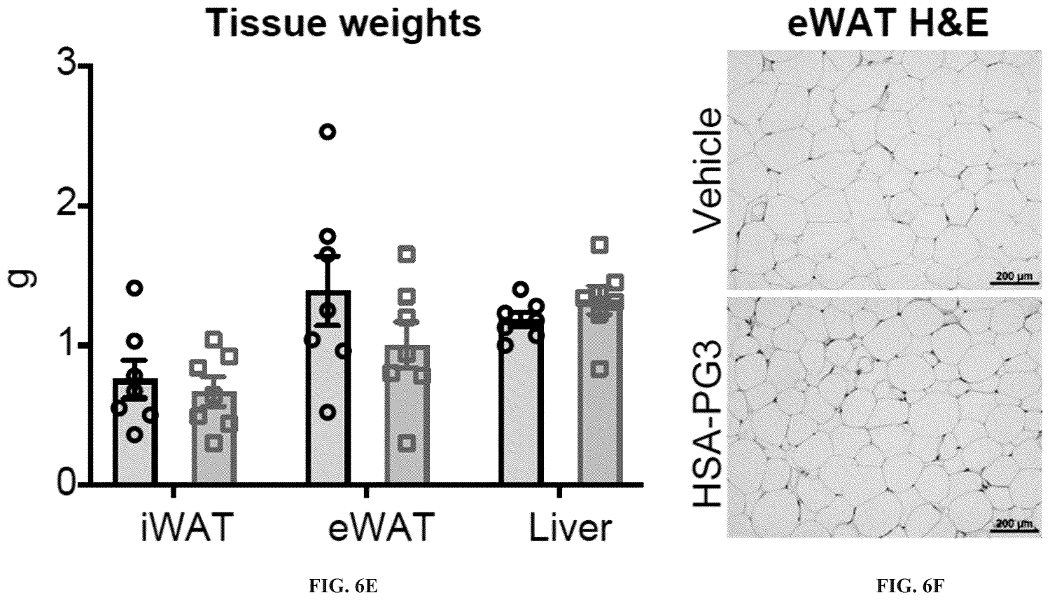

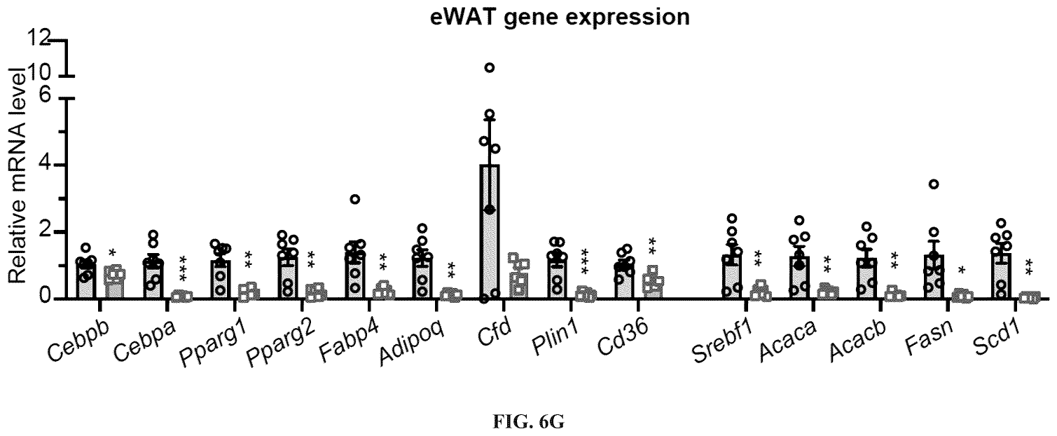

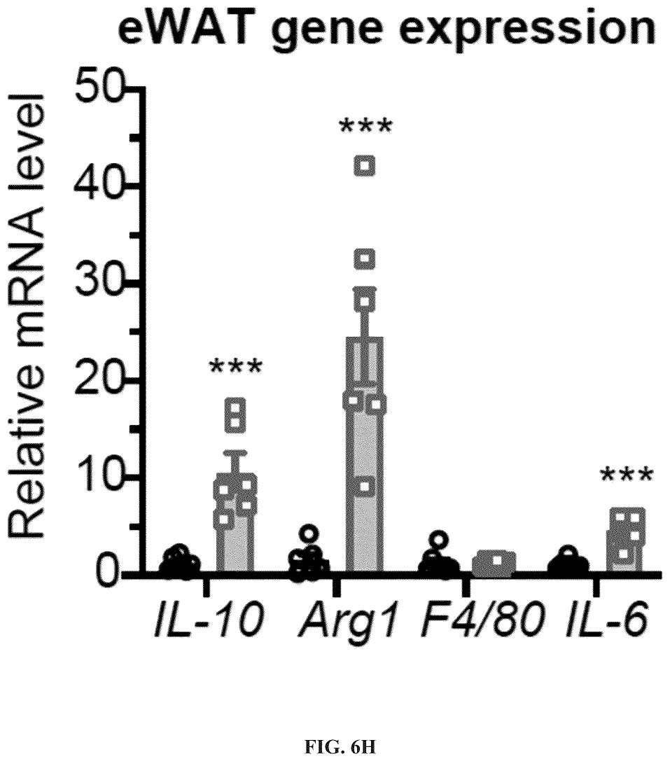

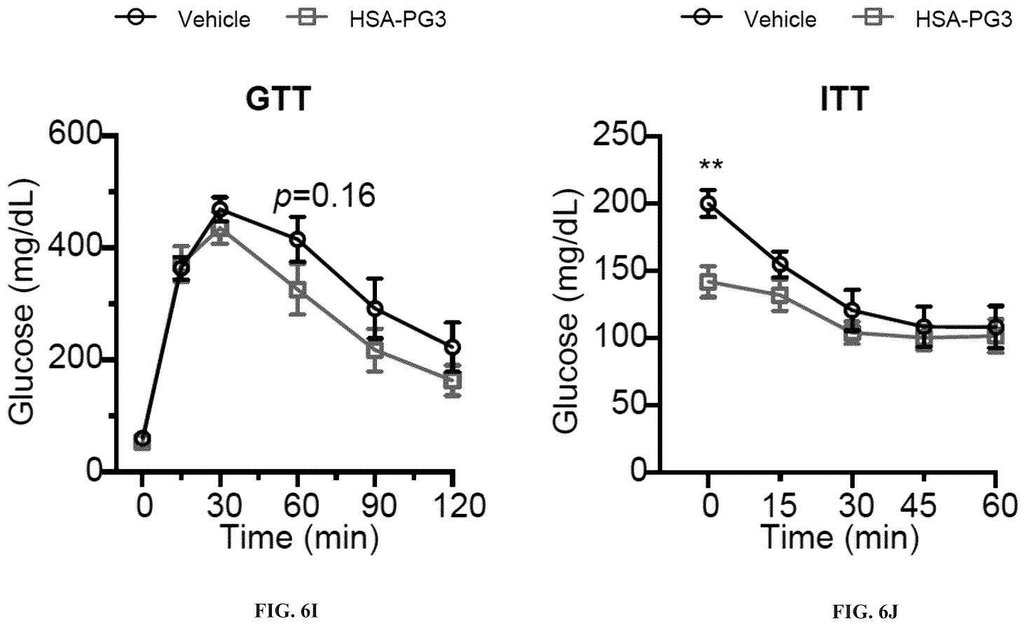

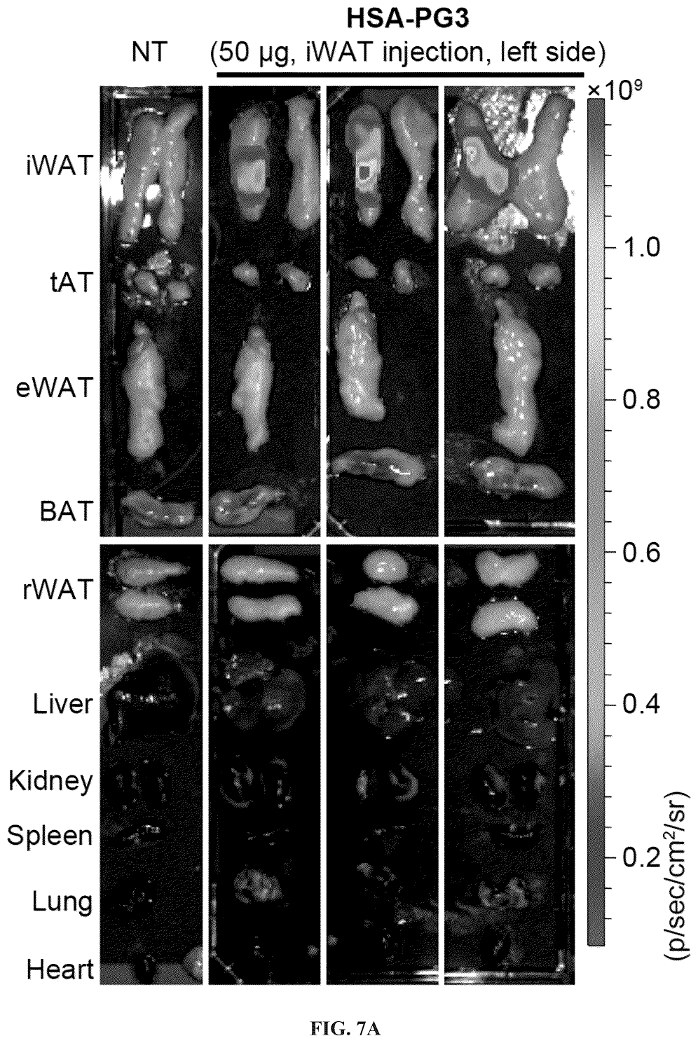

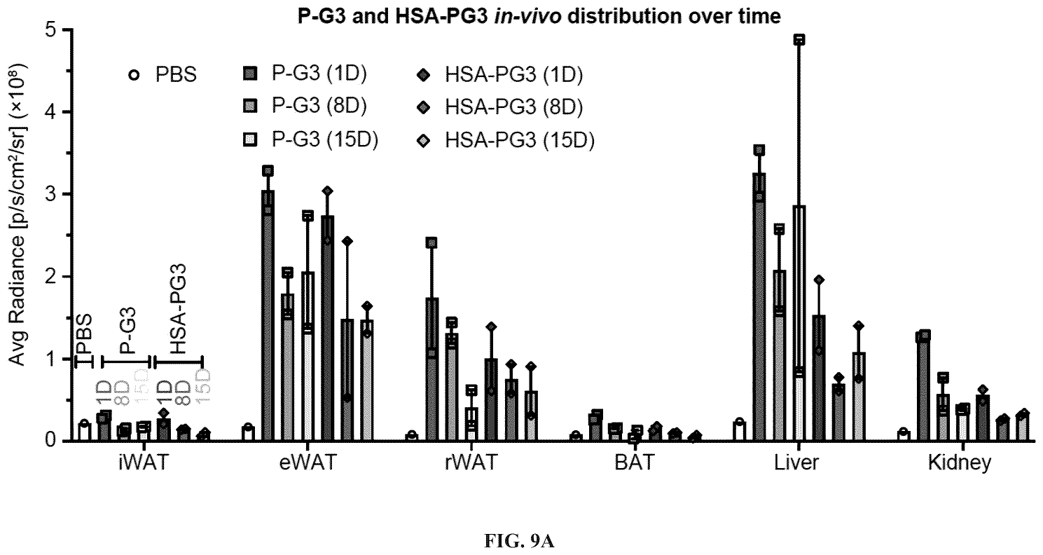

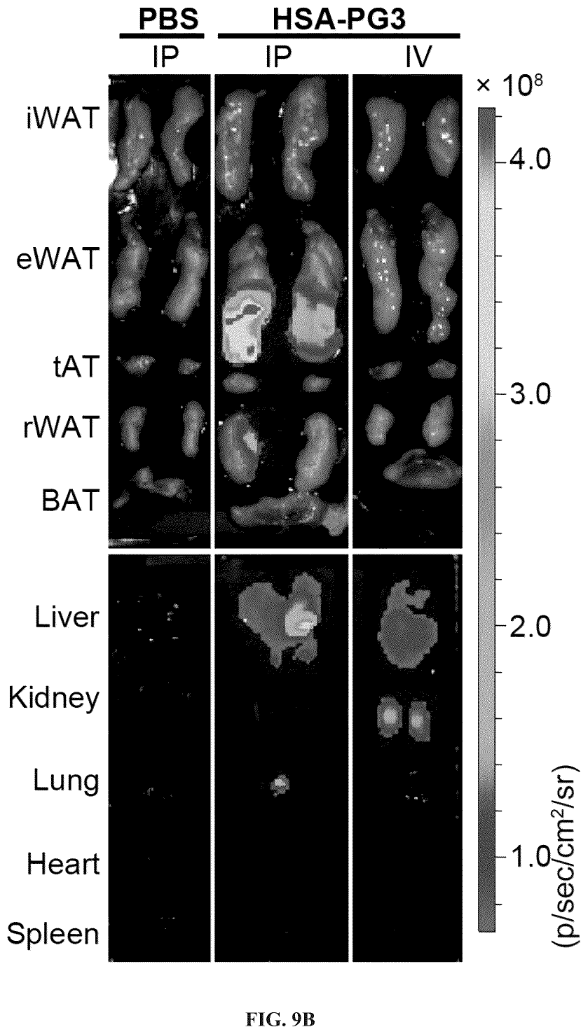

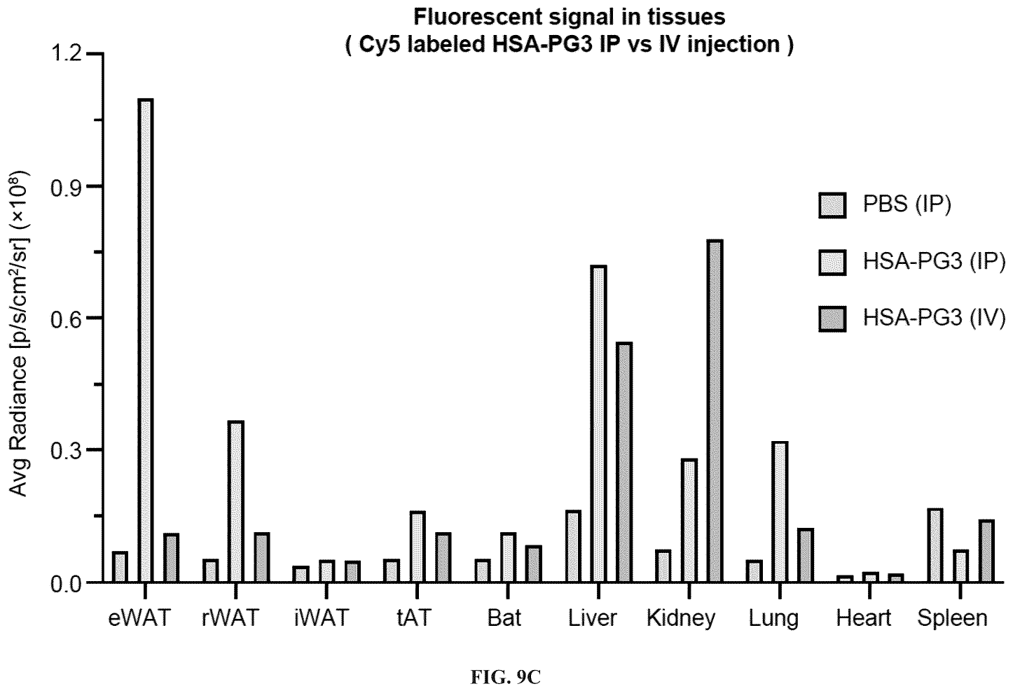

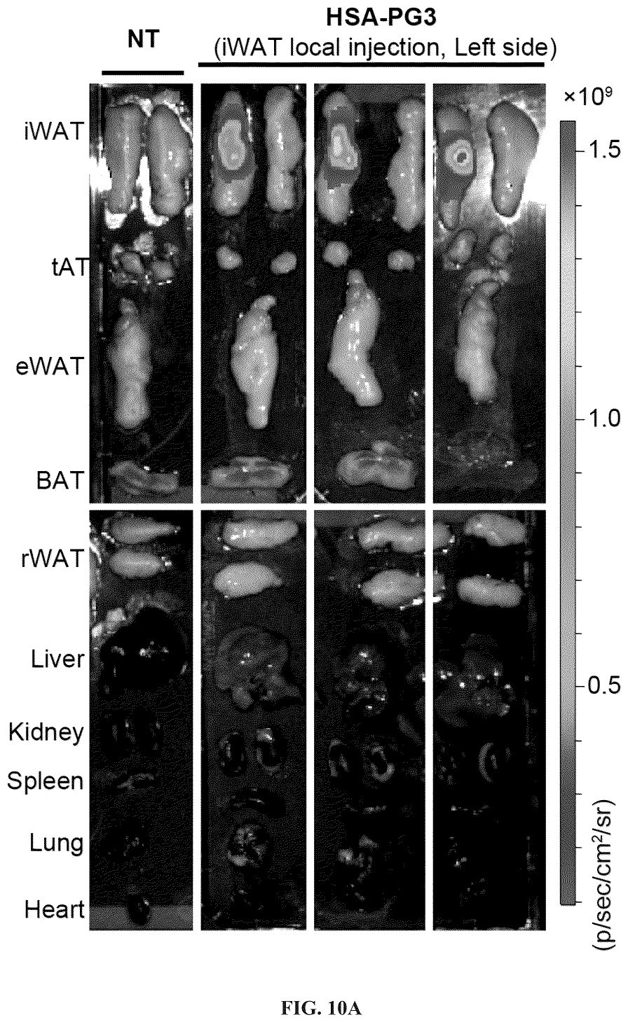

Next, the in vivo function of HSA-PG3 complexes in treating obesity were assessed. We first compared the biodistribution of HSA-PG3 with that of P-G3. Both showed efficient systemic distribution to the liver, kidney, and the visceral fat depots eWAT and retroperitoneal WAT (rWAT) via IP injection (FIG. 6A and FIG. 9A). However, over time, a lower signal of HSA-PG3 was detected in the liver, while the presence in visceral fat depots persisted at 8- and 15-days post-injection. In addition, this visceral biodistribution of HSA-PG3 was sensitive to the administration route on whether it was IP or intravenous (IV) injection (FIGS. 9B, 9C). We then treated C57BL/6 mice with HSA-PG3 via IP injection twice a week at a P-G3 dose of 10 μg/g BW and monitored their body weight change on HFD feeding (FIG. 5B). HSA-PG3 showed the delayed inhibition of body weight increase, gradually reaching approximately 10% less weight gain than controls after 7.5 weeks of treatment (FIG. 5C, D). The inhibition of fat mass mainly occurred in eWAT and it was modest (FIG. 5E). Despite the barely affected adipocyte size, there was a strong inhibition of adipogenic genes and lipogenic genes by HSA-PG3 in eWAT (FIG. 5F, G). Again, the anti-inflammatory markers IL-10 and Arg1 were markedly stimulated by HSA-PG3 in eWAT without changing the macrophage infiltration marker F4/80 (FIG. 5H), reinforcing its potential to alleviate chronic inflammation in obesity. Furthermore, glucose intolerance and insulin resistance induced by the HFD challenge were both improved by HSA-PG3 treatment (FIGS. 6I, 6J). Collectively, the HSA-PG3 complex showed similar anti-obesity effects and metabolic benefits compared to P-G3, although the effects were generally milder, likely due to a different pharmacodynamic effect because of the sustained release.

### 4.6. Reduction of Focal Subcutaneous Adiposity by HSA-PG3

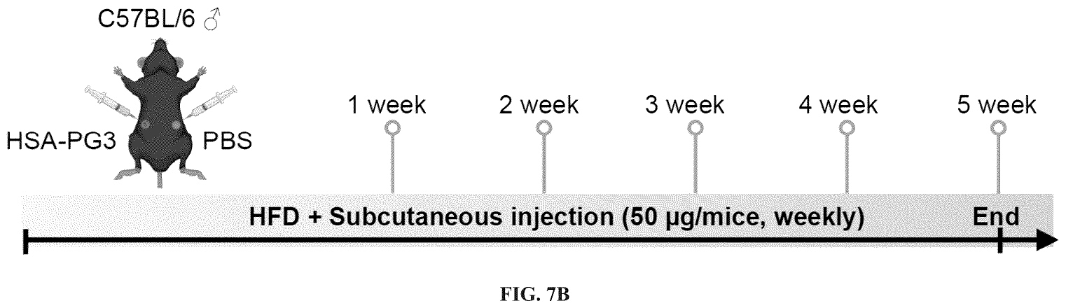

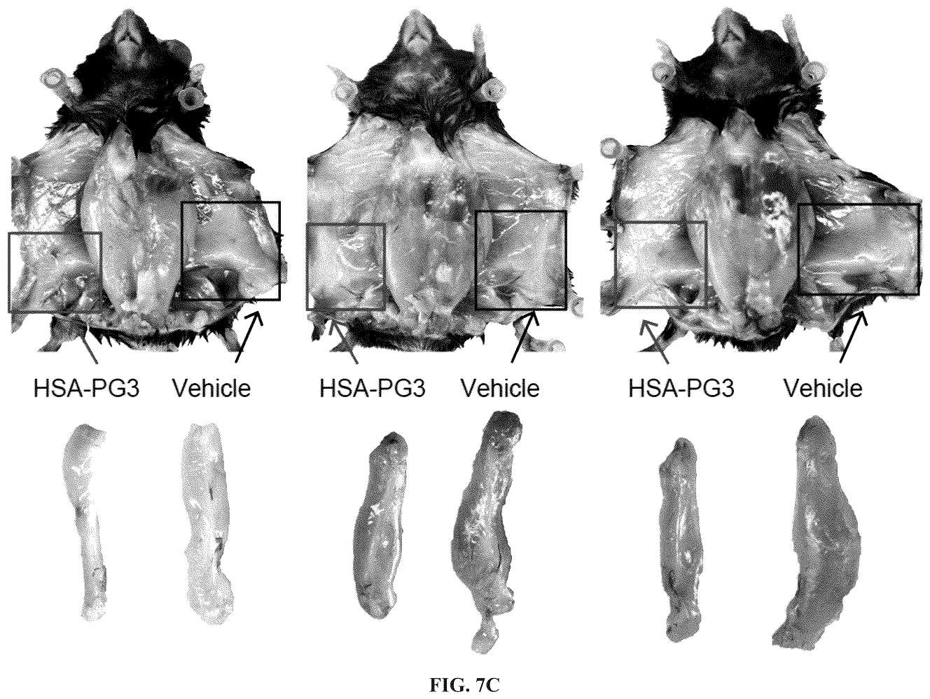

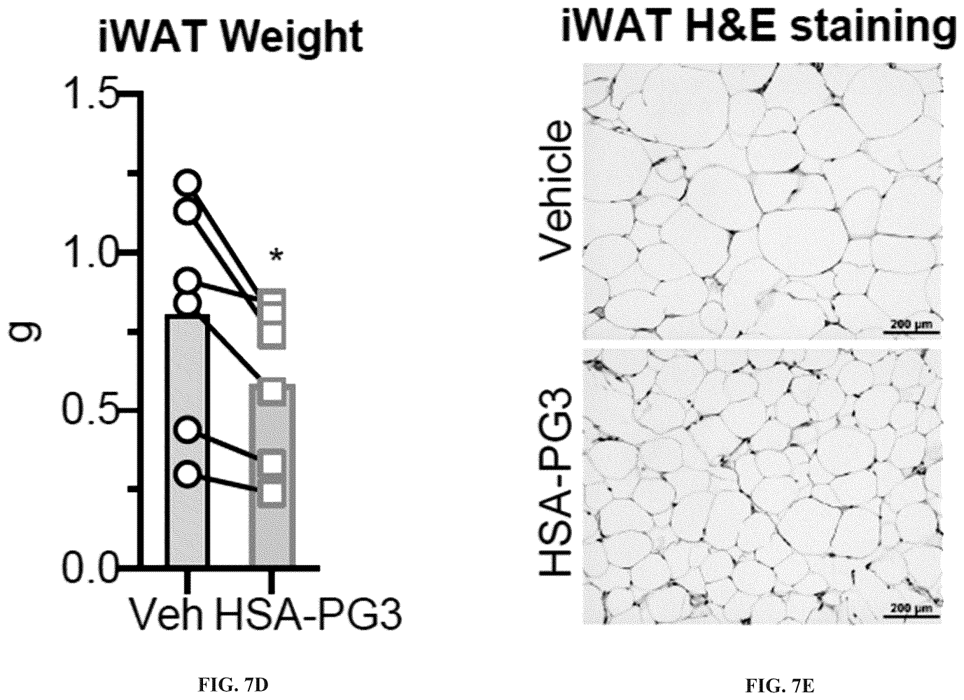

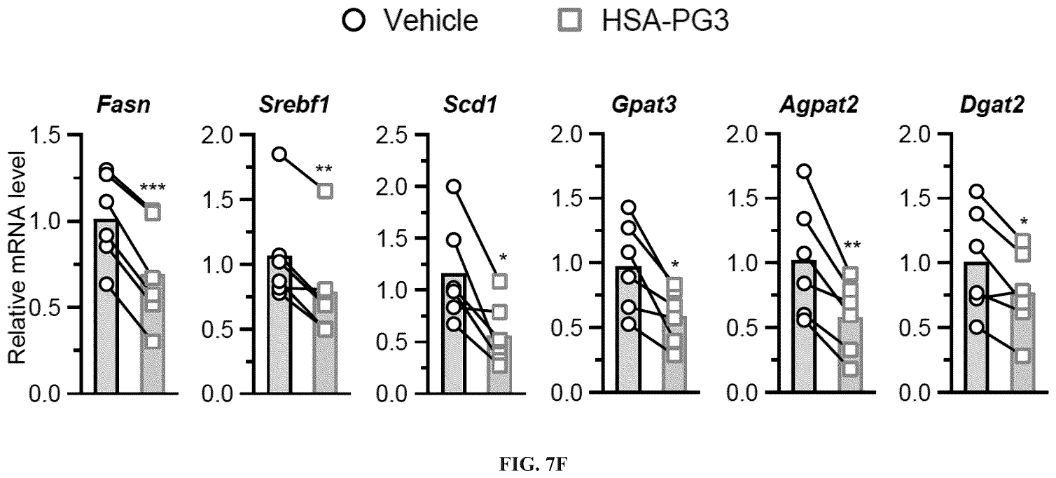

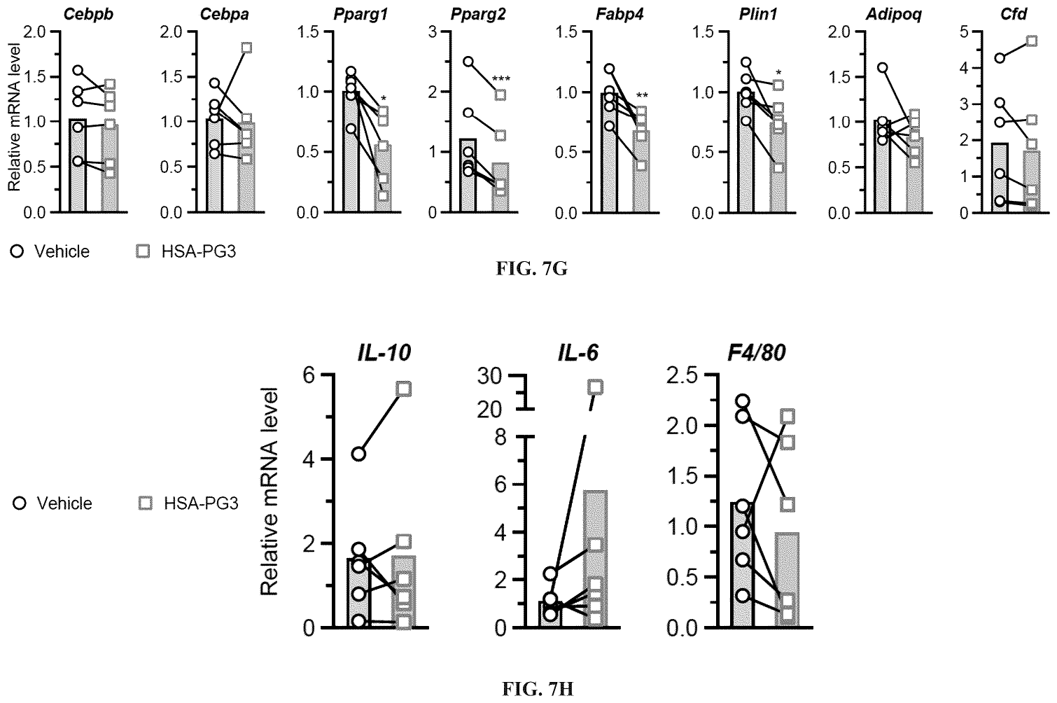

Depot-specific fat reduction is an even greater and more important challenge than weight loss per se, as all current obesity interventions, whether targeting energy homeostasis or food absorption, lack depot specificity. Several procedures reduce focal subcutaneous adiposity (e.g., liposuction) but most are based on a destructive strategy. Given the controlled release of P-G3 from the HSA complex and its strong repression of lipid synthetic genes, we investigated whether HSA-PG3 could be used to overcome the challenge of focal adiposity treatment. To this end, we locally injected HSA-PG3 into iWAT, a major subcutaneous fat depot in mice, and examined its biodistribution. Cy5-labeled HSA-PG3 was restricted to the iWAT on the injected side without distributing to the iWAT on the other side or any other organs examined, including the adjacent thigh adipose tissue (tAT)[42](FIG. 7A). The local specificity of the fluorescent signal was strictly retained at least 7 days post-injection (FIGS. 10A-10C). Hence, we devised a weekly treatment of 50 μg of HSA-PG3 in one side of the iWAT of DIO mice (FIG. 7B), estimating an equivalent P-G3 dose of 10 μg/g fat mass/day based on the average of 0.7 g iWAT depot size. Treatment of HSA-PG3 for 5 weeks significantly reduced the iWAT mass compared with the vehicle-treated side in the same mouse (FIG. 7C), with an average of a 30% reduction (FIG. 7D). In further support, the adipocyte size in the treated side was reduced (FIG. 7E). Underlying the smaller adipocyte size is the prevalent repression of genes involved in lipid synthesis, including de novo fatty acid synthesis (Fasn, Srebf1, and Scd1) and triglyceride synthesis (Gpat3, Agpat2, and Dgat2) (FIG. 7F). Interestingly, adipocyte functions appear to be largely maintained given the normal expression of housekeeping adipokine genes (Adipoq and Cfd) and adipogenic transcription factors (Cebpa and Cebpb), while some genes related to lipid storage (Pparg1, Pparg2, Fabp4, and Plin1) were downregulated (FIG. 7G). Moreover, the local inhibition of fat mass did not cause an increase in F4/80, a marker for macrophage infiltration and inflammation (FIG. 7H). Of note, HSA alone did not reduce iWAT depot size nor repress most lipid synthetic genes (FIGS. 10D, 10E). However, it did downregulate Pparg1, Pparg2, and Fabp4 (FIG. 10F), suggesting the P-G3-dependent repression of lipogenesis. Taken together, HSA-complexed P-G3, as a complex, displays the potential to treat focal adiposity in a targeted and safer manner.

### 5. Methods

In one aspect, provided herein is a method of treating accumulated fat in a patient in need thereof, comprising administering into the accumulated fat a therapeutically effective amount of P-G3 and/or HAS-P-G3 or a pharmaceutically acceptable salt thereof and at least one pharmaceutically acceptable excipient, wherein the accumulated fat results from or causes one or more of excess axillary fat, lateral periaxillary fat, pre axillary fat, post axillary fat, anterior periaxillary fat, posterior periaxillary fat, fat on the upper back, bra fat, back of arm fat, fat on the anterolateral flank, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including, but not limited to periumbilical fat, fat above periumbilical area, fat below periumbilical area, or any combination of two or more thereof), excess fat on the buttocks, mons pubis fat, excess fat around the ankles, fat on the upper back of the thigh, excess fat on the foot, pseudogynocomastia fat, lipoma, lipodystrophy (such as Dunning-type lipodystrophy), lipomatosis such as familial multiple lipomatosis, post-liposuction fat deposits, and obstructive sleep apnea. In some embodiments, the administering step is conducted by subcutaneous injection.

In another aspect, provided herein is a method of treating accumulated fat in a patient in need thereof, comprising administering into the accumulated fat a therapeutically effective amount of P-G3 and/or HSA-PG3 or a pharmaceutically acceptable salt thereof and at least one pharmaceutically acceptable excipient, wherein the accumulated fat is one or more of excess axillary fat, lateral periaxillary fat, pre axillary fat, post axillary fat, anterior periaxillary fat, posterior periaxillary fat, fat on the upper back, bra fat, back of arm fat, fat on the anterolateral flank, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including, but not limited to periumbilical fat, fat above periumbilical area, fat below periumbilical area, or any combination of two or more thereof), excess fat on the buttocks, mons pubis fat, excess fat around the ankles, fat on the upper back of the thigh, excess fat on the foot, pseudogynocomastia fat, and post-liposuction fat deposits. In some embodiments, the administering step is conducted by subcutaneous injection.

In another aspect, provided herein is a method of reducing accumulated fat in a patient in need thereof, comprising administering into the accumulated fat a therapeutically effective amount of P-G3 and/or HSA-PG3 or a pharmaceutically acceptable salt thereof and at least one pharmaceutically acceptable excipient, wherein the accumulated fat results from or causes one or more of excess axillary fat, lateral periaxillary fat, pre axillary fat, post axillary fat, anterior periaxillary fat, posterior periaxillary fat, fat on the upper back, bra fat, back of arm fat, fat on the anterolateral flank, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including, but not limited to periumbilical fat, fat above periumbilical area, fat below periumbilical area, or any combination of two or more thereof), excess fat on the buttocks, mons pubis fat, excess fat around the ankles, fat on the upper back of the thigh, excess fat on the foot, pseudogynocomastia fat, lipoma, lipodystrophy (such as Dunning-type lipodystrophy), lipomatosis such as familial multiple lipomatosis, post-liposuction fat deposits, and obstructive sleep apnea. In some embodiments, the administering step is conducted by subcutaneous injection.

In another aspect, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes one or more of excess axillary fat, lateral periaxillary fat, pre axillary fat, post axillary fat, anterior periaxillary fat, posterior periaxillary fat, fat on the upper back, bra fat, back of arm fat, fat on the anterolateral flank, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including, but not limited to periumbilical fat, fat above periumbilical area, fat below periumbilical area, or any combination of two or more thereof), excess fat on the buttocks, mons pubis fat, excess fat around the ankles, fat on the upper back of the thigh, excess fat on the foot, pseudogynocomastia fat, lipoma, lipodystrophy (such as Dunning-type lipodystrophy), lipomatosis such as familial multiple lipomatosis, post-liposuction fat deposits, and obstructive sleep apnea. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes bra fat (including, but not limited to, one or more of excess axillary fat, lateral periaxillary fat, pre axillary fat, post axillary fat, anterior periaxillary fat, posterior periaxillary fat, and fat on the upper back). In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes excess axillary fat. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes periaxillary fat. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes lateral periaxillary fat. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes pre axillary fat. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes post axillary fat. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes fat on the upper back. In some embodiments, the administering step is conducted by subcutaneous injection.

In some embodiments, provided herein is a method of reducing bra fat in a patient in need thereof, comprising locally administering into the bra fat a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing excess axillary fat in a patient in need thereof, comprising locally administering into the excess axillary fat a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing periaxillary fat in a patient in need thereof, comprising locally administering into the periaxillary fat a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing lateral periaxillary fat in a patient in need thereof, comprising locally administering into the lateral periaxillary fat a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing pre axillary fat in a patient in need thereof, comprising locally administering into the pre axillary fat a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing post axillary fat in a patient in need thereof, comprising locally administering into the post axillary fat a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing posterior periaxillary fat in a patient in need thereof, comprising locally administering into the posterior periaxillary fat a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing anterior periaxillary axillary fat in a patient in need thereof, comprising locally administering into the anterior periaxillary axillary fat a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing fat on the upper back in a patient in need thereof, comprising locally administering into the fat on the upper back a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes back of arm fat. In some embodiments, the administering step is conducted by subcutaneous injection.

In some embodiments, provided herein is a method of reducing back of arm fat in a patient in need thereof, comprising locally administering into the back of arm fat a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes a love handle. In some embodiments, the administering step is conducted by subcutaneous injection.

In some embodiments, provided herein is a method of reducing a love handle in a patient in need thereof, comprising locally administering into the love handle a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes fat on the anterolateral flank. In some embodiments, the administering step is conducted by subcutaneous injection.

In some embodiments, provided herein is a method of reducing fat on the anterolateral flank in a patient in need thereof, comprising locally administering into the fat on the anterolateral flank a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes excess fat at the sides of the waistline of the patient. In some embodiments, the administering step is conducted by subcutaneous injection.

In some embodiments, provided herein is a method of reducing deposits of excess fat at the sides of the waistline in a patient in need thereof, comprising locally administering into the deposits of excess fat at the sides of the waistline a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes medial knee fat. In some embodiments, the administering step is conducted by subcutaneous injection.

In some embodiments, provided herein is a method of reducing medial knee fat in a patient in need thereof, comprising locally administering into the medial knee fat a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes inner upper thigh fat. In some embodiments, the administering step is conducted by subcutaneous injection.

In some embodiments, provided herein is a method of reducing inner upper thigh fat in a patient in need thereof, comprising locally administering into the inner upper thigh fat a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes outer upper thigh fat. In some embodiments, the administering step is conducted by subcutaneous injection.

In some embodiments, provided herein is a method of reducing outer upper thigh fat in a patient in need thereof, comprising locally administering into the outer upper thigh fat a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes calf fat. In some embodiments, the administering step is conducted by subcutaneous injection.

In some embodiments, provided herein is a method of reducing calf fat in a patient in need thereof, comprising locally administering into the calf fat a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes fat around the ankles. In some embodiments, the administering step is conducted by subcutaneous injection.

In some embodiments, provided herein is a method of reducing fat around the ankles in a patient in need thereof, comprising locally administering into the fat around the ankles a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes excess fat on the face. In some embodiments, the administering step is conducted by subcutaneous injection.

In some embodiments, provided herein is a method of reducing excess fat on the face in a patient in need thereof, comprising locally administering into the excess fat on the face a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes intraorbital fat. In some embodiments, the administering step is conducted by subcutaneous injection.

In some embodiments, provided herein is a method of reducing intraorbital fat in a patient in need thereof, comprising locally administering into the intraorbital fat a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes periorbital fat. In some embodiments, the administering step is conducted by subcutaneous injection.

In some embodiments, provided herein is a method of reducing periorbital fat in a patient in need thereof, comprising locally administering into the periorbital fat a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes malar fat and/or jaw fat. In some embodiments, the administering step is conducted by subcutaneous injection.

In some embodiments, provided herein is a method of reducing malar fat and/or jaw fat in a patient in need thereof, comprising locally administering into the malar fat and/or jaw fat a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes stomach fat (including, but not limited to periumbilical fat, fat above periumbilical area, fat below periumbilical area, or any combination of two or more thereof). In some embodiments, the administering step is conducted by subcutaneous injection. In some embodiments, stomach fat is referred to as fat above and below periumbilical area. As used herein, the term “fat above and below periumbilical area” refers to fat above and below the belly button.

In some embodiments, provided herein is a method of reducing stomach fat in a patient in need thereof, comprising locally administering into the stomach fat a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection. In some embodiments, stomach fat is referred to as fat above and below periumbilical area.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes periumbilical fat. In some embodiments, the administering step is conducted by subcutaneous injection.

In some embodiments, provided herein is a method of reducing periumbilical fat in a patient in need thereof, comprising locally administering into the periumbilical fat a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes fat above periumbilical area. In some embodiments, the administering step is conducted by subcutaneous injection. As used herein, the term “fat above periumbilical area” refers to fat above the belly button or fat on the upper stomach.

In some embodiments, provided herein is a method of reducing fat above periumbilical area in a patient in need thereof, comprising locally administering into the fat above periumbilical area a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes fat below periumbilical area. In some embodiments, the administering step is conducted by subcutaneous injection. As used herein, the term “fat below periumbilical area” refers to fat below the belly button or fat on the lower stomach.

In some embodiments, provided herein is a method of reducing fat below periumbilical area in a patient in need thereof, comprising locally administering into the fat below periumbilical area a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes excess fat on the back or buttocks. In some embodiments, the administering step is conducted by subcutaneous injection.

In some embodiments, provided herein is a method of reducing excess fat on the back in a patient in need thereof, comprising locally administering into the excess fat on the back a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing excess fat on the buttocks in a patient in need thereof, comprising locally administering into the excess fat on buttocks a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes mons pubis fat. In some embodiments, the administering step is conducted by subcutaneous injection.

In some embodiments, provided herein is a method of reducing mons pubis fat in a patient in need thereof, comprising locally administering into the mons pubis fat a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes fat on the upper back of the thigh. In some embodiments, the administering step is conducted by subcutaneous injection.

In some embodiments, provided herein is a method of reducing fat on the upper back of the thigh in a patient in need thereof, comprising locally administering into the fat on the upper back of the thigh a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes excess fat on the foot. In some embodiments, the administering step is conducted by subcutaneous injection.

In some embodiments, provided herein is a method of reducing excess fat on the foot in a patient in need thereof, comprising locally administering into the excess fat on the foot a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes pseudogynocomastia fat. In some embodiments, the administering step is conducted by subcutaneous injection.

In some embodiments, provided herein is a method of reducing pseudogynocomastia fat in a patient in need thereof, comprising locally administering into the pseudogynocomastia fat a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes lipoma. In some embodiments, the administering step is conducted by subcutaneous injection.

In some embodiments, provided herein is a method of reducing a lipoma in a patient in need thereof, comprising locally administering into the lipoma a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes lipodystrophy (such as Dunning-type lipodystrophy). In some embodiments, the administering step is conducted by subcutaneous injection.

In some embodiments, provided herein is a method of treating lipodystrophy (such as Dunning-type lipodystrophy) in a patient in need thereof, comprising local administration of a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes lipomatosis such as familial multiple lipomatosis. In some embodiments, the administering step is conducted by subcutaneous injection.

In some embodiments, provided herein is a method of treating lipomatosis such as familial multiple lipomatosis in a patient in need thereof, comprising local administration of a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes post-liposuction fat deposits. In some embodiments, the administering step is conducted by subcutaneous injection.

In some embodiments, provided herein is a method of reducing post-liposuction fat deposits in a patient in need thereof, comprising locally administering into the post-liposuction fat deposits a composition comprising or consisting essentially of a therapeutically effective amount of P-G3 and/or HSA-PG3 or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes obstructive sleep apnea. In some embodiments, the administering step is conducted by subcutaneous injection.

In another aspect, provided herein is a method of reducing accumulated fat in a patient in need thereof, comprising administering into the accumulated fat by local injection a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes one or more of excess axillary fat, lateral periaxillary fat, pre axillary fat, post axillary fat, anterior periaxillary fat, posterior periaxillary fat, fat on the upper back, bra fat, back of arm fat, fat on the anterolateral flank, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including, but not limited to periumbilical fat, fat above periumbilical area, fat below periumbilical area, or any combination of two or more thereof), excess fat on the buttocks, mons pubis fat, excess fat around the ankles, fat on the upper back of the thigh, excess fat on the foot, pseudogynocomastia fat, lipoma, lipodystrophy (such as Dunning-type lipodystrophy), lipomatosis such as familial multiple lipomatosis, post-liposuction fat deposits, and obstructive sleep apnea. In some embodiments, the administering step is conducted by subcutaneous injection.

In another aspect, provided herein is a method of reducing accumulated fat in a patient in need thereof, comprising administering into the accumulated fat by local subcutaneous injection a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes one or more of excess axillary fat, lateral periaxillary fat, pre axillary fat, post axillary fat, anterior periaxillary fat, posterior periaxillary fat, fat on the upper back, bra fat, back of arm fat, fat on the anterolateral flank, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including, but not limited to periumbilical fat, fat above periumbilical area, fat below periumbilical area, or any combination of two or more thereof), excess fat on the buttocks, mons pubis fat, excess fat around the ankles, fat on the upper back of the thigh, excess fat on the foot, pseudogynocomastia fat, lipoma, lipodystrophy (such as Dunning-type lipodystrophy), lipomatosis such as familial multiple lipomatosis, post-liposuction fat deposits, and obstructive sleep apnea.

In another aspect, provided herein is a method of reducing accumulated fat in a patient in need thereof, comprising administering into the accumulated fat by a plurality of injections (i.e., two or more injections) a composition comprising or consisting essentially of a therapeutically effective amount of P-G3 and/or HSA-PG3 or a pharmaceutically acceptable salt thereof and at least one pharmaceutically acceptable excipient, wherein the accumulated fat results from or causes one or more of excess axillary fat, lateral periaxillary fat, pre axillary fat, post axillary fat, anterior periaxillary fat, posterior periaxillary fat, fat on the upper back, bra fat, back of arm fat, fat on the anterolateral flank, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including, but not limited to periumbilical fat, fat above periumbilical area, fat below periumbilical area, or any combination of two or more thereof), excess fat on the buttocks, mons pubis fat, excess fat around the ankles, fat on the upper back of the thigh, excess fat on the foot, pseudogynocomastia fat, lipoma, lipodystrophy (such as Dunning-type lipodystrophy), lipomatosis such as familial multiple lipomatosis, post-liposuction fat deposits, and obstructive sleep apnea.

In another aspect, provided herein is a cosmetic method of reducing accumulated fat in a subject in need thereof, comprising administering into the accumulated fat by subcutaneous injection a composition comprising or consisting essentially of P-G3 and/or HSA-PG3, or a salt thereof, and at least one cosmetically acceptable excipient, wherein the accumulated fat results from or causes one or more of excess axillary fat, lateral periaxillary fat, pre axillary fat, post axillary fat, anterior periaxillary fat, posterior periaxillary fat, fat on the upper back, bra fat, back of arm fat, fat on the anterolateral flank, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including, but not limited to periumbilical fat, fat above periumbilical area, fat below periumbilical area, or any combination of two or more thereof), excess fat on the back or buttocks, mons pubis fat, excess fat around the ankles, fat on the upper back of the thigh, excess fat on the foot, pseudogynocomastia fat, lipoma, lipomatosis such as familial multiple lipomatosis, and post-liposuction fat deposits.

In another aspect, provided herein is a method of correcting one or more post-interventional treatment contour irregularities in a subject in need thereof, comprising administering to the subject a composition described herein. Post-interventional treatment contour irregularities include, but are not limited to, contour irregularities following one or more of plastic surgery, breast augmentation, reconstruction, mammopexy, fat injection, liposuction, energy-based treatment (including, but not limited to cryotherapy, radio frequency treatment, laser treatment), and breast reduction (male or female).

In another aspect, provided herein is a method of correcting one or more post-interventional treatment contour irregularities in a subject in need thereof, comprising administering to the subject a composition comprising or consisting essentially of a therapeutically effective amount of P-G3 and/or HSA-PG3 or a pharmaceutically acceptable salt thereof and at least one pharmaceutically acceptable or cosmetically acceptable excipient.

In another aspect, provided herein is a method of correcting one or more post-interventional treatment contour irregularities in a subject in need thereof, comprising administering to the subject a composition comprising or consisting essentially of a therapeutically effective amount of P-G3 and/or HSA-PG3 or a pharmaceutically acceptable salt thereof and at least one pharmaceutically acceptable or cosmetically acceptable excipient, wherein the one or more contour irregularities result from plastic surgery, breast augmentation, reconstruction, mammopexy, fat injection, liposuction, energy-based treatment (including, but not limited to cryotherapy, radio frequency treatment, laser treatment), breast reduction (male or female), or a combination of two or more thereof.

In another aspect, provided herein is a method of correcting one or more post-interventional treatment contour irregularities in a subject in need thereof, comprising locally administering to the subject a composition comprising or consisting essentially of a therapeutically effective amount of P-G3 and/or HSA-PG3 or a pharmaceutically acceptable salt thereof and at least one pharmaceutically acceptable or cosmetically acceptable excipient, wherein the one or more contour irregularities result from plastic surgery, breast augmentation, reconstruction, mammopexy, fat injection, liposuction, energy-based treatment (including, but not limited to cryotherapy, radio frequency treatment, laser treatment), breast reduction (male or female), or a combination of two or more thereof.

In another aspect, provided herein is a method of correcting one or more post-interventional treatment contour irregularities in a subject in need thereof, comprising locally administering to the subject by one or more subcutaneous injections a composition comprising or consisting essentially of a therapeutically effective amount of P-G3 and/or HSA-PG3 or a pharmaceutically acceptable salt thereof and at least one pharmaceutically acceptable or cosmetically acceptable excipient, wherein the one or more contour irregularities result from plastic surgery, breast augmentation, reconstruction, mammopexy, fat injection, liposuction, energy-based treatment (including, but not limited to cryotherapy, radio frequency treatment, laser treatment), breast reduction (male or female), or a combination of two or more thereof.

In another aspect, provided herein is a method of correcting one or more post-interventional treatment contour irregularities in a subject in need thereof, comprising locally administering to the subject by a plurality of subcutaneous injections a composition comprising or consisting essentially of a therapeutically effective amount of P-G3 and/or HSA-PG3 or a pharmaceutically acceptable salt thereof and at least one pharmaceutically acceptable or cosmetically acceptable excipient, wherein the one or more contour irregularities result from plastic surgery, breast augmentation, reconstruction, mammopexy, fat injection, liposuction, energy-based treatment (including, but not limited to cryotherapy, radio frequency treatment, laser treatment), breast reduction (male or female), or a combination of two or more thereof.

In another aspect, provided herein is a cosmetic method of correcting one or more post-interventional treatment contour irregularities in a subject in need thereof, comprising locally administering to the subject a composition comprising or consisting essentially of an effective amount of P-G3 and/or HSA-PG3 or a pharmaceutically acceptable salt thereof and at least one cosmetically acceptable excipient, wherein the one or more contour irregularities result from plastic surgery, breast augmentation, reconstruction, mammopexy, fat injection, liposuction, energy-based treatment (including, but not limited to cryotherapy, radio frequency treatment, laser treatment), breast reduction (male or female), or a combination of two or more thereof.

In another aspect, provided herein is a cosmetic method of correcting one or more post-interventional treatment contour irregularities in a subject in need thereof, comprising locally administering to the subject by one or more subcutaneous injections a composition comprising or consisting essentially of an effective amount of P-G3 and/or HSA-PG3 or a pharmaceutically acceptable salt thereof and at least one cosmetically acceptable excipient, wherein the one or more contour irregularities result from plastic surgery, breast augmentation, reconstruction, mammopexy, fat injection, liposuction, energy-based treatment (including, but not limited to cryotherapy, radio frequency treatment, laser treatment), breast reduction (male or female), or a combination of two or more thereof.

In another aspect, provided herein is a cosmetic method of correcting one or more post-interventional treatment contour irregularities in a subject in need thereof, comprising locally administering to the subject by a plurality of subcutaneous injections a composition comprising or consisting essentially of an effective amount of P-G3 and/or HSA-PG3 or a pharmaceutically acceptable salt thereof and at least one cosmetically acceptable excipient, wherein the one or more contour irregularities result from plastic surgery, breast augmentation, reconstruction, mammopexy, fat injection, liposuction, energy-based treatment (including, but not limited to cryotherapy, radio frequency treatment, laser treatment), breast reduction (male or female), or a combination of two or more thereof.

The present subject matter being thus described, it will be apparent that the same may be modified or varied in many ways. Such modifications and variations are not to be regarded as a departure from the spirit and scope of the present subject matter, and all such modifications and variations are intended to be included within the scope of the following claims.

### REFERENCES

ADDIN EN.REFLIST [1] W. H. Organization, Obesity and overweight. <https://www.who.int/news-room/fact-sheets/detail/obesity-and-overweight>).
[2] I. Mahmoud, A. S. Al-Wandi, S. S. Gharaibeh, S. A. Mohamed, Concordances and correlations between anthropometric indices of obesity: a systematic review, Public Health 198 (2021) 301-306.
[3] S. P. Weisberg, D. McCann, M. Desai, M. Rosenbaum, R. L. Leibel, A. W. Ferrante, Jr., Obesity is associated with macrophage accumulation in adipose tissue, J Clin Invest 112(12) (2003) 1796-808.
[4] H. Xu, G. T. Barnes, Q. Yang, G. Tan, D. Yang, C. J. Chou, J. Sole, A. Nichols, J. S. Ross, L. A. Tartaglia, H. Chen, Chronic inflammation in fat plays a crucial role in the development of obesity-related insulin resistance, J Clin Invest 112(12) (2003) 1821-30.
[5] A. Chawla, K. D. Nguyen, Y. P. Goh, Macrophage-mediated inflammation in metabolic disease, Nat Rev Immunol 11(11) (2011) 738-49.
[6] M. S. Burhans, D. K. Hagman, J. N. Kuzma, K. A. Schmidt, M. Kratz, Contribution of Adipose Tissue Inflammation to the Development of Type 2 Diabetes Mellitus, Compr Physiol 9(1) (2018) 1-58.
[7] P. Trayhurn, Hypoxia and adipose tissue function and dysfunction in obesity, Physiol Rev 93(1) (2013) 1-21.
[8] G. H. Goossens, E. E. Blaak, Adipose tissue dysfunction and impaired metabolic health in human obesity: a matter of oxygen?, Front Endocrinol (Lausanne) 6 (2015) 55.
[9] J. Ye, Z. Gao, J. Yin, Q. He, Hypoxia is a potential risk factor for chronic inflammation and adiponectin reduction in adipose tissue of ob/ob and dietary obese mice, Am J Physiol Endocrinol Metab 293(4) (2007) E1118-28.
[10] M. Kuroda, H. Sakaue, Adipocyte Death and Chronic Inflammation in Obesity, J Med Invest 64(3.4) (2017) 193-196.
[11] S. Heinonen, J. Buzkova, M. Muniandy, R. Kaksonen, M. Ollikainen, K. Ismail, A. Hakkarainen, J. Lundbom, N. Lundbom, K. Vuolteenaho, E. Moilanen, J. Kaprio, A. Rissanen, A. Suomalainen, K. H. Pietilainen, Impaired Mitochondrial Biogenesis in Adipose Tissue in Acquired Obesity, Diabetes 64(9) (2015) 3135-45.
[12] S. Park, A. Aintablian, B. Coupe, S. G. Bouret, The endoplasmic reticulum stress-autophagy pathway controls hypothalamic development and energy balance regulation in leptin-deficient neonates, Nat Commun 11(1) (2020) 1914.
[13] N. K. Sharma, S. K. Das, A. K. Mondal, O. G. Hackney, W. S. Chu, P. A. Kern, N. Rasouli, H. J. Spencer, A. Yao-Borengasser, S. C. Elbein, Endoplasmic reticulum stress markers are associated with obesity in nondiabetic subjects, J Clin Endocrinol Metab 93(11) (2008) 4532-41.
[14] M. F. Gregor, L. Yang, E. Fabbrini, B. S. Mohammed, J. C. Eagon, G. S. Hotamisligil, S. Klein, Endoplasmic reticulum stress is reduced in tissues of obese subjects after weight loss, Diabetes 58(3) (2009) 693-700.
[15] G. Boden, X. Duan, C. Homko, E. J. Molina, W. Song, O. Perez, P. Cheung, S. Merali, Increase in endoplasmic reticulum stress-related proteins and genes in adipose tissue of obese, insulin-resistant individuals, Diabetes 57(9) (2008) 2438-44.
[16] P. M. Ridker, B. M. Everett, T. Thuren, J. G. MacFadyen, W. H. Chang, C. Ballantyne, F. Fonseca, J. Nicolau, W. Koenig, S. D. Anker, J. J. P. Kastelein, J. H. Cornel, P. Pais, D. Pella, J. Genest, R. Cifkova, A. Lorenzatti, T. Forster, Z. Kobalava, L. Vida-Simiti, M. Flather, H. Shimokawa, H. Ogawa, M. Dellborg, P. R. F. Rossi, R. P. T. Troquay, P. Libby, R. J. Glynn, C. T. Group, Antiinflammatory Therapy with Canakinumab for Atherosclerotic Disease, N Engl J Med 377(12) (2017) 1119-1131.
[17] C. G. Perry, A. Spiers, S. J. Cleland, G. D. Lowe, J. R. Petrie, J. M. Connell, Glucocorticoids and insulin sensitivity: dissociation of insulin's metabolic and vascular actions, J Clin Endocrinol Metab 88(12) (2003) 6008-14.
[18] D. Preiss, S. R. Seshasai, P. Welsh, S. A. Murphy, J. E. Ho, D. D. Waters, D. A. DeMicco, P. Barter, C. P. Cannon, M. S. Sabatine, E. Braunwald, J. J. Kastelein, J. A. de Lemos, M. A. Blazing, T. R. Pedersen, M. J. Tikkanen, N. Sattar, K. K. Ray, Risk of incident diabetes with intensive-dose compared with moderate-dose statin therapy: a meta-analysis, JAMA 305(24) (2011) 2556-64.
[19] H. Dominguez, H. Storgaard, C. Rask-Madsen, T. Steffen Hermann, N. Ihlemann, D. Baunbjerg Nielsen, C. Spohr, L. Kober, A. Vaag, C. Torp-Pedersen, Metabolic and vascular effects of tumor necrosis factor-alpha blockade with etanercept in obese patients with type 2 diabetes, J Vasc Res 42(6) (2005) 517-25.
[20] F. Ofei, S. Hurel, J. Newkirk, M. Sopwith, R. Taylor, Effects of an engineered human anti-TNF-alpha antibody (CDP571) on insulin sensitivity and glycemic control in patients with NIDDM, Diabetes 45(7) (1996) 881-5.
[21] N. Paquot, M. J. Castillo, P. J. Lefebvre, A. J. Scheen, No increased insulin sensitivity after a single intravenous administration of a recombinant human tumor necrosis factor receptor: Fc fusion protein in obese insulin-resistant patients, J Clin Endocrinol Metab 85(3) (2000) 1316-9.
[22] A. Rosenvinge, R. Krogh-Madsen, B. Baslund, B. K. Pedersen, Insulin resistance in patients with rheumatoid arthritis: effect of anti-TNFalpha therapy, Scand J Rheumatol 36(2) (2007) 91-6.
[23] J. Lee, J. W. Sohn, Y Zhang, K. W. Leong, D. Pisetsky, B. A. Sullenger, Nucleic acid-binding polymers as anti-inflammatory agents, Proc Natl Acad Sci USA 108(34) (2011) 14055-60.
[24] J. Lee, J. G. Jackman, J. Kwun, M. Manook, A. Moreno, E. A. Elster, A. D. Kirk, K. W. Leong, B. A. Sullenger, Nucleic acid scavenging microfiber mesh inhibits trauma-induced inflammation and thrombosis, Biomaterials 120 (2017) 94-102.
[25] C. Yang, J. Dawulieti, K. B. Zhang, C. X. Cheng, Y. W. Zhao, H. Z. Hu, M. Li, M. Zhang, L. Chen, K. W. Leong, D. Shao, An Injectable Antibiotic Hydrogel that Scavenges Proinflammatory Factors for the Treatment of Severe Abdominal Trauma, Adv Funct Mater (2022).
[26] E. K. Holl, K. L. Shumansky, L. B. Borst, A. D. Burnette, C. J. Sample, E. A. Ramsburg, B. A. Sullenger, Scavenging nucleic acid debris to combat autoimmunity and infectious disease, Proc Natl Acad Sci USA 113(35) (2016) 9728-33.
[27] H. Liang, B. Peng, C. Dong, L. Liu, J. Mao, S. Wei, X. Wang, H. Xu, J. Shen, H. Q. Mao, X. Gao, K. W. Leong, Y Chen, Cationic nanoparticle as an inhibitor of cell-free DNA-induced inflammation, Nat Commun 9(1) (2018) 4291.
[28] B. Peng, H. Liang, Y Li, C. Dong, J. Shen, H. Q. Mao, K. W. Leong, Y Chen, L. Liu, Tuned Cationic Dendronized Polymer: Molecular Scavenger for Rheumatoid Arthritis Treatment, Angew Chem Int Ed Engl 58(13) (2019) 4254-4258.
[29] H. Shen, B. Xu, C. Yang, W. Xue, Z. You, X. Wu, D. Ma, D. Shao, K. Leong, J. Dai, A DAMP-scavenging, IL-10-releasing hydrogel promotes neural regeneration and motor function recovery after spinal cord injury, Biomaterials 280 (2022) 121279.
[30] C. Shi, J. Dawulieti, F. Shi, C. Yang, Q. Qin, T. Shi, L. Wang, H. Hu, M. Sun, L. Ren, F. Chen, Y Zhao, F. Liu, M. Li, L. Mu, D. Liu, D. Shao, K. W. Leong, J. She, A nanoparticulate dual scavenger for targeted therapy of inflammatory bowel disease, Sci Adv 8(4) (2022) eabj2372.
[31] J. Dawulieti, M. Sun, Y Zhao, D. Shao, H. Yan, Y. H. Lao, H. Hu, L. Cui, X. Lv, F. Liu, C. W. Chi, Y Zhang, M. Li, M. Zhang, H. Tian, X. Chen, K. W. Leong, L. Chen, Treatment of severe sepsis with nanoparticulate cell-free DNA scavengers, Sci Adv 6(22) (2020) eaay7148.
[32] F. Liu, S. Sheng, D. Shao, YQ. Xiao, Y. L. Zhong, J. Zhou, C. H. Quek, Y. B. Wang, J. Dawulieti, C. Yang, H. Y. Tian, X. S. Chen, K. W. Leong, Targeting multiple mediators of sepsis using multifunctional tannic acid-Zn2+-gentamicin nanoparticles, Matter-Us 4(11) (2021) 3677-3695.
[33] F. Liu, S. Sheng, D. Shao, Y. Q. Xiao, Y. L. Zhong, J. Zhou, C. H. Quek, Y. B. Wang, Z. M. Hu, H. S. Liu, Y. H. Li, H. Y. Tian, K. W. Leong, X. S. Chen, A Cationic Metal-Organic Framework to Scavenge Cell-Free DNA for Severe Sepsis Management, Nano Lett 21(6) (2021) 2461-2469.
[34] Z. Tu, Y Zhong, H. Hu, D. Shao, R. Haag, M. Schirner, J. Lee, B. Sullenger, K. W. Leong, Design of therapeutic biomaterials to control inflammation, Nat Rev Mater 7(7) (2022) 557-574.
[35] D. Li, F. Zhang, X. Zhang, C. Xue, M. Namwanje, L. Fan, M. P. Reilly, F. Hu, L. Qiang, Distinct functions of PPARgamma isoforms in regulating adipocyte plasticity, Biochem Biophys Res Commun 481(1-2) (2016) 132-138.
[36] I. Tinoco, Jr., C. Bustamante, How RNA folds, J Mol Biol 293(2) (1999) 271-81.
[37] D. Patsouris, J. K. Reddy, M. Muller, S. Kersten, Peroxisome proliferator-activated receptor alpha mediates the effects of high-fat diet on hepatic gene expression, Endocrinology 147(3) (2006) 1508-16.
[38] M. Guerre-Millo, C. Rouault, P. Poulain, J. Andre, V. Poitout, J. M. Peters, F. J. Gonzalez, J. C. Fruchart, G. Reach, B. Staels, PPAR-alpha-null mice are protected from high-fat diet-induced insulin resistance, Diabetes 50(12) (2001) 2809-14.
[39] F. Kratz, Albumin as a drug carrier: design of prodrugs, drug conjugates and nanoparticles, J Control Release 132(3) (2008) 171-83.
[40] P. Ma, R. J. Mumper, Paclitaxel Nano-Delivery Systems: A Comprehensive Review, J Nanomed Nanotechnol 4(2) (2013) 1000164.
[41] D. A. Yardley, nab-Paclitaxel mechanisms of action and delivery, J Control Release 170(3) (2013) 365-72.
[42] M. Chan, Y. C. Lim, J. Yang, M. Namwanje, L. Liu, L. Qiang, Identification of a natural beige adipose depot in mice, J Biol Chem 294(17) (2019) 6751-6761.
[43] J. J. Bernard, C. Cowing-Zitron, T. Nakatsuji, B. Muehleisen, J. Muto, A. W. Borkowski, L. Martinez, E. L. Greidinger, B. D. Yu, R. L. Gallo, Ultraviolet radiation damages self noncoding RNA and is detected by TLR3, Nat Med 18(8) (2012) 1286-90.
[44] S. Nishimoto, D. Fukuda, Y Higashikuni, K. Tanaka, Y Hirata, C. Murata, J. R. Kim-Kaneyama, F. Sato, M. Bando, S. Yagi, T. Soeki, T. Hayashi, I. Imoto, H. Sakaue, M. Shimabukuro, M. Sata, Obesity-induced DNA released from adipocytes stimulates chronic adipose tissue inflammation and insulin resistance, Sci Adv 2(3) (2016) e1501332.
[45] F. Abedi-Gaballu, G. Dehghan, M. Ghaffari, R. Yekta, S. Abbaspour-Ravasjani, B. Baradaran, J. E. N. Dolatabadi, M. R. Hamblin, PAMAM dendrimers as efficient drug and gene delivery nanosystems for cancer therapy, Appl Mater Today 12 (2018) 177-190.
[46] R. Duncan, L. Izzo, Dendrimer biocompatibility and toxicity, Adv Drug Deliv Rev 57(15) (2005) 2215-37.
[47] J. B. Pryor, B. J. Harper, S. L. Harper, Comparative toxicological assessment of PAMAM and thiophosphoryl dendrimers using embryonic zebrafish, Int J Nanomedicine 9 (2014) 1947-56.
[48] D. Fischer, Y Li, B. Ahlemeyer, J. Krieglstein, T. Kissel, In vitro cytotoxicity testing of polycations: influence of polymer structure on cell viability and hemolysis, Biomaterials 24(7) (2003) 1121-31.
[49] L. M. Kaminskas, B. J. Boyd, C. J. Porter, Dendrimer pharmacokinetics: the effect of size, structure and surface characteristics on ADME properties, Nanomedicine (Lond) 6(6) (2011) 1063-84.
[50] M. Najlah, S. Freeman, M. Khoder, D. Attwood, A. D'Emanuele, In Vitro Evaluation of Third Generation PAMAM Dendrimer Conjugates, Molecules 22(10) (2017).
[51] M. Karimi, S. Bahrami, S. B. Ravari, P. S. Zangabad, H. Mirshekari, M. Bozorgomid, S. Shahreza, M. Sori, M. R. Hamblin, Albumin nanostructures as advanced drug delivery systems, Expert Opin Drug Deliv 13(11) (2016) 1609-1623.
[52] N. Liu, Y Hao, Z. Yin, M. Ma, L. Wang, X. Zhang, Self-assembled human serum albumin-coated complexes for gene delivery with improved transfection, Pharmazie 67(2) (2012) 174-81.
[53] R. K. Tekade, M. Tekade, M. Kumar, A. S. Chauhan, Dendrimer-stabilized smart-nanoparticle (DSSN) platform for targeted delivery of hydrophobic antitumor therapeutics, Pharm Res 32(3) (2015) 910-28.
[54] R. V. Araujo, S. D. S. Santos, E. Igne Ferreira, J. Giarolla, New Advances in General Biomedical Applications of PAMAM Dendrimers, Molecules 23(11) (2018).

What is claimed:

1. A complex for treating accumulated fat in a subject, comprising:
human serum albumin (HSA); and
cationic polyamidoamine generation 3 (P-G3) in complex with the HSA, formed in microspheres, and configured for treating obesity, reducing body weight, and improving metabolic functions in the subject.

2. The complex of claim 1, wherein the microspheres have a diameter between 0.5 micrometer and 500 micrometers.

3. The complex of either of claims 1 and 2, wherein the complex has an HSA:P-G3 mass ratio of at least 5:1.

4. The complex of any of claims 1-3, wherein the complex has an HSA:P-G3 mass ratio of about 10:1.

5. The complex of any of claims 1-4, wherein the microspheres attain controlled release of the P-G3 into the subject.

6. A method of treating accumulated fat in a subject, the method comprising administering to the subject a therapeutically effective amount of
(a) the complex of any of claims 1-5 or
(b) a pharmaceutically acceptable salt of cationic P-G3 and at least one pharmaceutically acceptable excipient.

7. The method of claim 6, wherein the administration comprises enteral administration of the complex to the subject.

8. The method of claim 6, wherein the administration comprises parenteral administration of the complex to the subject.

9. The method of either of claims 6 or 8, wherein the administration comprises local injection into subcutaneous fat of the subject.

10. The method of any of claims 6, 8, and 9, wherein the administration comprises a plurality of injections.

11. The method of any of claims 6-10, wherein the method treats or reduces focal adiposity in the subject.

12. The method of any of claims 6-11, wherein the method ameliorates chronic inflammation associated with obesity in the subject.

13. The method of any of claims 6-12, wherein the method results in metabolic improvements in the subject.

14. The method of any of claims 6-13, wherein the complex scavenges cfDNA.

15. The method of any of claims 6-13, wherein the complex scavenges cfRNA.
